(19) United States
(12) Patent Application Publication
(10) Pub. No.: US 2007/0134688 A1
(43) Pub. Date: Jun. 14, 2007

(54) CALCULATED INDEX OF GENOMIC EXPRESSION OF ESTROGEN RECEPTOR (ER) AND ER-RELATED GENES

(75) Inventors: W. Fraser Symmans, Houston, TX (US); Christos Hatzis, Melrose, MA (US); Keith Anderson, Rochester, MN (US); Lajos Pusztai, Pearland, TX (US)

Correspondence Address: FULBRIGHT & JAWORSKI L.L.P., 600 CONGRESS AVE., SUITE 2400, AUSTIN, TX, 78701, US

(73) Assignees: THE BOARD OF REGENTS OF THE UNIVERSITY OF TEXAS SYSTEM; NUVERA BIOSCIENCES, INC.

(21) Appl. No.: 11/530,785

(22) Filed: Sep. 11, 2006

Related U.S. Application Data
(60) Provisional application No. 60/715,403, filed on Sep. 9, 2005.
(60) Provisional application No. 60/822,879, filed on Aug. 18, 2006.

(51) Int. Cl. C12Q 1/68; G01N 33/574; A61K 31/56

(52) U.S. Cl. 435/6; 435/7.23; 514/182

(57) ABSTRACT

The present invention provides the identification and combination of genes that are expressed in tumors that are responsive to a given therapeutic agent and whose combined expression can be used as an index that correlates with responsiveness to that therapeutic agent. One or more of the genes of the present invention may be used as markers (or surrogate markers) to identify tumors that are likely to be successfully treated by that agent or class of agents such as hormonal or endocrine treatment.

This application claims priority to U.S. Provisional Patent Applications Ser. No. 60/715,403, filed on Sep. 9, 2005 and Ser. No. 60/822,879 filed on Aug. 18, 2006, each of which is incorporated herein by reference in their entirety.

### I. FIELD OF THE INVENTION

The present invention relates to the fields of medicine and molecular biology, particularly transcriptional profiling, molecular arrays and predictive tools for response to cancer treatment.

### II. BACKGROUND

Endocrine treatments of breast cancer target the activity of estrogen receptor alpha (ER, gene name ESR1). The current challenges for treatment of patients with ER-positive breast cancer include the ability to predict benefit from endocrine (hormonal) therapy and/or chemotherapy, to select among endocrine agents, and to define the duration and sequence of endocrine treatments. These challenges are each conceptually related to the state of ER activity in a patient's breast cancer. Since ER acts principally at the level of transcriptional control, a genomic index to measure downstream ER-associated gene expression activity in a patient's tumor sample can help quantify ER pathway activity, and thus dependence on estrogen, and intrinsic sensitivity to endocrine therapy. Treatment-specific predictors can enable available multiplex genomic technology to provide a way to specifically address a distinct clinical decision or treatment choice.

### SUMMARY OF THE INVENTION

Embodiments of the invention include methods of calculating an index, e.g., an estrogen receptor (ER) reporter index or a sensitivity to endocrine treatment (SET) index, for assessing the hormonal sensitivity of a tumor comprising one or more of the steps of: (a) obtaining gene expression data from samples obtained from a plurality of patients; (b) calculating one or more reference gene expression profiles from a plurality of patients with a specific diagnosis, e.g., cancer diagnosis; (c) normalizing the expression data of additional samples to the reference gene expression profile; (d) measuring and reporting estrogen receptor (ER) gene expression from the profile as a method for defining ER status of a cancer; (e) identifying the genes to define a profile to measure ER-related transcriptional activity in any cancer sample; (f) defining one or more reference ER-related gene expression profiles; (g) calculating a weighted index or index (e.g., a SET index) based on ER-related gene expression in any patient sample(s) and the ER-related reference profile; and/or (h) combining the measurements of ER gene expression and the index (e.g., weighted index or SET index) for ER-related gene expression to measure and report the gene expression of ER and ER-related transcriptional profile as a continuous or categorical result. In certain aspects assessing the likely sensitivity of any cancer to treatment by measuring ER and ER-related gene expression singly or as a combined result. In certain embodiments, the cancer is suspected of being a hormone-sensitive cancer, preferably an estrogen-sensitive cancer. In certain aspects, the suspected estrogen-sensitive cancer is breast cancer. The ER-related genes may include one or more genes selected from two-hundred ER related genes or gene probes. In certain aspects of the invention, ER related genes or gene probes include 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 191, 192, 193, 194, 195, 196, 197, 198, 199, or 200 ER related genes or gene probes. In particular embodiments one or more genes are selected from Table 1 or Table 2. The weighted or calculated index may be based on similarity with the reference ER-related gene expression profile(s). In a further aspect of the invention similarity is calculated based on: (a) an algorithm to calculate a distance metric, such as one or a combination of Euclidian, Mahalanobis, or general Miknowski norms; and/or (b) calculation of a correlation coefficient for the sample based on expression levels or ranks of expression levels. The calculation of the weighted or reporter index may include various parameters (e.g., patient covariates) related to the disease condition including, but not limited to the parameters or characteristics of tumor size, nodal status, grade, age, and/or evaluation of prognosis based on distant relapse-free survival (DRFS) or overall survival (OS) of patients.

Embodiments of the invention include patients that are ER-positive and receiving hormonal therapy. In certain aspects the hormonal therapy includes, but is not limited to tamoxifen therapy and may include other known hormonal therapies used to treat cancers, particularly breast cancer. The treatment administered is typically a hormonal therapy, chemotherapy or a combination of the two. Additional aspects of the invention include evaluation of risk stratification of noncancerous cells and may be used to mitigate or prevent future disease. Still further aspects of the invention include normalization by a single digital standard. The method may further comprise normalizing expression data of the one or more samples to the ER-related gene expression profile. The expression data can be normalized to a digital standard. The digital standard can be a gene expression profile from a reference sample.

Further embodiments of the invention include methods of assessing patient sensitivity to treatment comprising one or more steps of: (a) determining expression levels of the ER gene and/or one or more additional ER-related genes; (b) calculating the value of the ER reporter index (e.g., a SET index); (c) assessing or predicting the response to hormonal therapy based on the value of the index; (d) assessing or predicting the response to an administered treatment (e.g., chemotherapy) based on the value of the index, and/or (e) selecting a treatment(s) for a patient based on consideration of the predicted responsiveness to hormonal therapy and/or chemotherapy.

In yet still further embodiments of the invention include a calculated index for predicting response (e.g., a response to treatment) produced by the method comprising the steps of: (a) obtaining gene expression data from samples obtained from a plurality of cancer patients; (b) normalizing the gene expression data; and (c) calculating an index (e.g., a weighted or SET index) based on the ER gene and one or more additional ER-related gene expression levels in the patient sample. In certain aspects the ER-related genes are selected as described supra. Parameters (e.g., patient covariates) used in conjunction with the calculation of the index includes, but is not limited to tumor size, nodal status, grade, age, evaluation of distant relapse-free survival (DRFS) or of overall survival (OS) of the patients and various combinations thereof. Typically, the patients are ER-positive and receiving hormonal therapy, preferably tamoxifen therapy. The methods of the invention may also include treatment administered as a combination of one or more cancer drugs. In particular aspects, the treatment administered is a hormonal therapy, a chemotherapy, or a combination of hormonal therapy and chemotherapy.

In yet still further embodiments of the invention include a calculated index for predicting response to therapy for late-stage (recurrent) cancer as performed by the method comprising the steps of: (a) obtaining gene expression data from samples obtained from a plurality of stage IV cancer patients; (b) normalizing the expression data; (c) calculating an index based on the ER gene and/or one or more additional ER-related gene expression levels in the patient sample; and (d) predicting response to therapy. Typically, the patients are ER-positive and have previously received, or are currently receiving hormonal therapy. The methods of the invention may also include treatment administered as a combination of one or more cancer drugs. In particular aspects, the treatment administered is a hormonal therapy, a chemotherapy, or a combination of hormonal therapy and chemotherapy.

Other embodiments of the invention include methods of assessing, e.g., assessing quantitatively, the estrogen receptor (ER) status of a cancer sample by measuring transcriptional activity comprising two or more of the steps of: (a) obtaining a sample of cancerous tissue from a patient; (b) determining mRNA gene expression levels of the ER gene in the sample; (c) establishing a cut-off ER mRNA value from the distribution of ER transcripts in a plurality of cancer samples, and/or (d) assessing ER status based on the mRNA level of the ER gene in the sample relative to the pre-determined cut-off level of mRNA transcript. The sample may be a biopsy sample, a surgically excised sample, a sample of bodily fluids, a fine needle aspiration biopsy, core needle biopsy, tissue sample, or exfoliative cytology sample. In certain aspects, the patient is a cancer patient, a patient suspected of having hormone-sensitive cancer, a patient suspected of having an estrogen or progesterone sensitive cancer, and/or a patient having or suspected of having breast cancer. In further aspects of the invention, the expression levels of the genes are determined by hybridization, nucleic amplification, or array hybridization, such as nucleic acid array hybridization. In certain aspects the nucleic acid array is a microarray. In still further embodiments, nucleic acid amplification is by polymerase chain reaction (PCR).

Embodiments of the invention may also include kits for the determination of ER status of cancer comprising: (a) reagents for determining expression levels of the ER gene and/or one or more additional ER-related genes in a sample; and/or (b) algorithm and software encoding the algorithm for calculating an ER reporter index from expression of ER and ER-related genes in a sample to determine the sensitivity of a patient to hormonal therapy.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specific embodiments presented herein.

FIG. 1. Selection probabilities P_{g}(50), P_{g}(100), P_{g}(200) for the 200 top-ranking probe sets in terms of their Spearman's rank correlation with the ESR1 transcript (probe set 205225_at) plotted as a function of the probe set's rank in the original dataset. Probabilities were estimated from 1000 bootstrap samples of the original dataset.

FIG. 2. Distribution of ranks of the top 200 genes estimated from 1000 bootstrap replications of the original dataset as a function of the magnitude of the Spearman's rank correlation with the ESR1 transcript.

FIGS. 3A-3D. Distribution of the index of expression of the 200 ER-related genes by ER status for (FIG. 3A) 277 tamoxifen-treated patients and (FIG. 3B) 286 node-negative untreated patients. (FIGS. 3C and 3D) Dependence of ER gene expression index on ESR1 mRNA expression for patient populations corresponding to panels (FIG. 3A) and (FIG. 3B).

FIG. 4. Replicate measurements of ESR1 expression, PGR expression, ER reporter index and sensitivity to endocrine treatment (SET) index in 35 sample pairs of experimental replicates using residual RNA. Also shown is the 45° line through the origin. FIG. 4A (ESR1), FIG. 4B (PGR), FIG. 4C (ER Reporter Index), and FIG. 4D (SET Index).

FIGS. 5A-5C. Predicted marginal risk of distant relapse at 10 years in ER-positive breast cancer patients treated with adjuvant tamoxifen as a continuous function of genomic covariates: (FIG. 5A) ESR1 (ER) expression level, (FIG. 5B) log-transformed PGR expression level, and (FIG. 5C) genomic sensitivity to endocrine therapy (SET) index. The dashed lines show the 95% confidence interval of the predicted risk rates.

FIGS. 6A-6D. Kaplan-Meier estimates of relapse-free survival in ER-positive patients treated with adjuvant tamoxifen (FIG. 6A, FIG. 6C) or in patients not receiving systemic therapy after surgery (FIG. 6B, FIG. 6D). Groups were defined by the SET index (FIG. 6A, FIG. 6B) or the median-dichotomized log-transformed PGR expression (FIG. 6C, FIG. 6D). P-values are from the log-rank test.

FIGS. 7A-7B. Kaplan-Meier estimates of relapse-free survival in ER-positive patients treated with adjuvant tamoxifen grouped by nodal status: (FIG. 7A) node-negative group; (FIG. 7B) node-positive group. P-values are from the log-rank test.

FIG. 8A-8D. Box plots demonstrate genomic measurements in 351 ER-positive samples categorized by AJCC Stage (58 stage I, 123 stage IIA, 107 stage IIB, 44 stage III, and 18 stage IV). Each box indicates the median and interquartile range, and the whisker lines extend 1.5× the interquartile range above the 75th percentile and below the 25th percentile. FIG. 8A=SET index; FIG. 8B=ESR1; FIG. 8C=Log PGR; FIG. 8D=GAPDH.

### DETAILED DESCRIPTION OF THE INVENTION

It has already been established that the overall transcriptional profile in breast cancers is dependent on ER status, being largely determined in ER-positive breast cancer by the genomic activity of ER on the transcription of numerous genes (Perou et al., 2000; van't Veer et al., 2002; Gruvberger et al., 2001; Pusztai et al., 2003). The inventors contemplate that the amount of ER-associated reporter gene expression is an indicator of ER transcriptional activity, likely dependence on ER activity, and sensitivity to hormonal therapy. Differences in expression of ER mRNA (the receptor) and ER reporter genes (the transcriptional output) might contribute to variable response of patients with ER-positive breast cancers to hormonal therapy (Buzdar, 2001; Howell and Dowsett, 2004; Hess et al, 2003). Herein, a set of genes are defined that are co-expressed with ER from an independent public database of Affymetrix U133A gene profiles from 286 lymph node-negative breast cancers and calculated an index score for their expression (Wang et al., 2005). Another goal was to determine whether the expression level of ESR1 gene, and value of this index for expression of ER reporter (associated) genes, is associated with distant relapse-free survival (DRFS) in other patients following adjuvant hormonal therapy with tamoxifen.

There are four main approaches to improving the ability to predict responsiveness to endocrine therapies. One approach is a standard predictive or chemopredictive study focused on treatment, in which a sufficiently powered discovery population of subjects is used to define a predictive test that must then be proven to be accurate in a similarly sized validation population (Ransohoff, 2005; Ransohoff 2004). Several studies have used this approach to define predictive genes for adjuvant tamoxifen therapy (Ma et al., 2004; Jansen et al., 2005; Loi et al., 2005). There are advantages to this approach, particularly when samples are available from mature studies for retrospective analysis. But two disadvantages are that the study design is empirical and that adjuvant treatment introduces surgery as a confounding variable, because it is impossible to ever know which patients were cured by their surgery and would never relapse, irrespective of their sensitivity to systemic therapy. Neoadjuvant chemotherapy trials enable a direct comparison of tumor characteristics with pathologic response (Ayers et al., 2004). While an empirical study design is needed for chemopredictive studies of cytotoxic chemotherapy regimens because multiple cellular pathways are likely to be disrupted, endocrine therapy of breast cancer specifically targets ER-mediated tumor growth and survival. The compositions and methods of the present invention may define and measure this ER-mediated effect supplanting the need for a limited empirical study design.

A second approach is to identify genes that are downregulated in vivo after treatment with an endocrine agent. This involves a small sample size of patients who undergo repeat biopsies, but is complicated by the selection of agent and dose used, variable timing of downregulation of different genes after therapy, and variable treatment effect in different tumors.

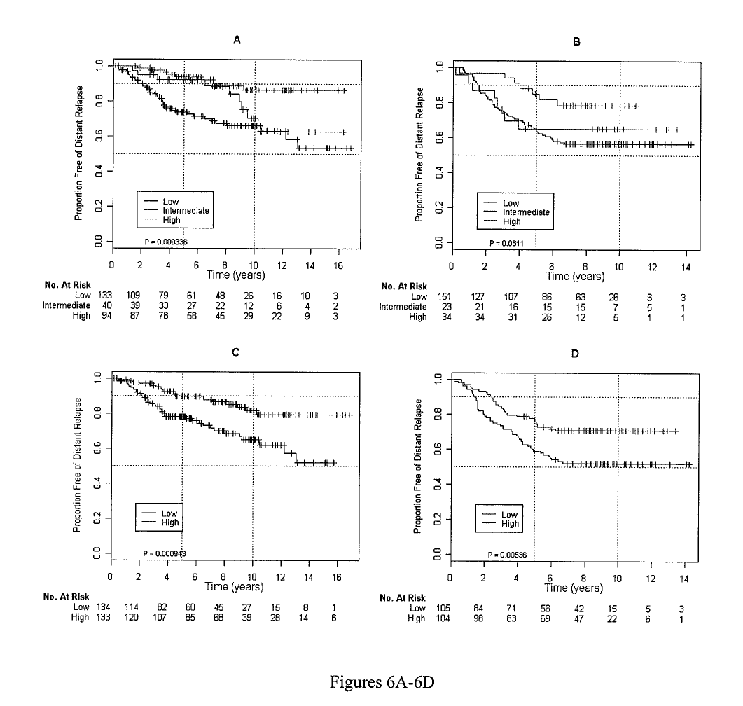

A third approach is to quantify receptor expression as accurately as possible. Semiquantitative scoring of ER immunoflourescent/immunohistochemical (IFIC) staining is related to disease-free survival following adjuvant tamoxifen (Harvey et al., 1999). For example, measurement of 16 selected genes (mostly related to ER, proliferation, and HER-2) using RT-PCR in a central reference laboratory predicts survival of women with tamoxifen-treated node-negative breast cancer (Paik et al., 2004). In a recent report, measurement of ER mRNA using RT-PCR diagnoses ER IHC status with 93% overall accuracy (Esteva et al., 2005). It was also recently reported that ER mRNA measurements from the same RT-PCR assay predict survival after adjuvant tamoxifen (Paik et al., 2005). So, if gene expression microarrays can reliably measure ER mRNA in a way that can be standardized in different laboratories, those measurements should predict response to endocrine treatment. Certain aspects of the invention described herein demonstrate that measurements of ER mRNA expression levels from microarrays also predict distant relapse-free survival following adjuvant tamoxifen therapy (Tables 4 and 5, and FIG. 6). However, other gene expression measurements from the microarray are informative as well.

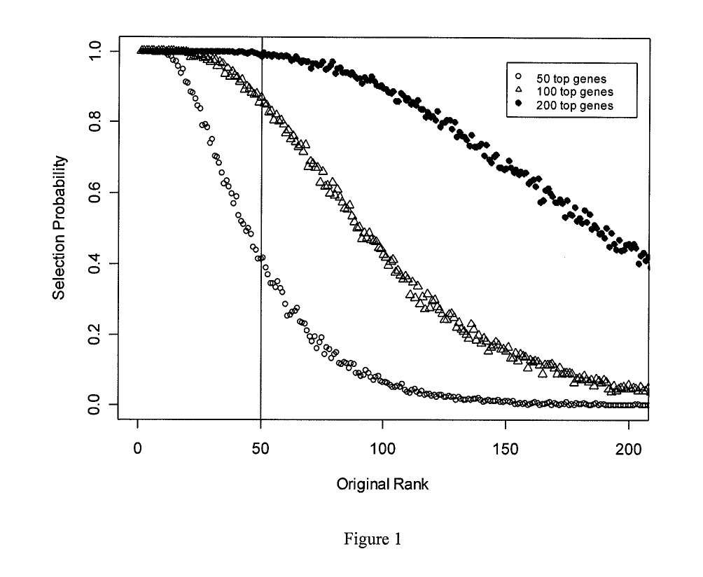

A fourth approach, selected by the inventors, measures ER gene expression and the transcriptional output from ER activity, taking advantage of the high-throughput microarray platform. This approach theoretically applies to all endocrine treatments and does not require the empirical discovery and validation study populations. If a continuous scale of endocrine responsiveness exists, then specific endocrine treatments could be matched to likely response. Some patients would have an excellent response from tamoxifen, but others may need more potent endocrine treatment to respond to the same extent. A challenge with this approach is to accurately define the number and correct ER reporter genes to measure. The approach was to define ER reporter genes from a large, independent data set of 286 breast cancer profiles from Affymetrix U133A arrays. It is not necessary that these patients receive endocrine treatment, or to know their immunohistochemical ER status or survival, in order to define the genes most correlated with ER gene expression. Even with the relatively large sample size of 286 cases, the inventors calculated that 200 genes should be included as reporter genes in order to contain the 50 most ER-related genes with 98.5% confidence and the 100 most related genes with about 90% confidence (FIG. 1). This demonstrates the importance of a sufficiently large reporter gene set to capture a reliable transcriptional signature for ER activity in breast cancers (Perou et al., 2000; Van't Veer et al., 2002; Gruvberger et al., 2001; Pusztai et al., 2003).

If quantitative measurements of the ER-related expression, expression of ER mRNA, and/or ER activity (represented by a calculated index of ER reporter gene expression) accurately predict benefit from hormonal therapy, it is possible to develop a continuous genomic scale of measurement for ER expression and activity. This scale could be used to identify subsets of patients with ER-positive breast cancer that: (1) are expected to benefit from tamoxifen alone, (2) require more potent endocrine therapy, (3) may require chemotherapy along with endocrine therapy, or (4) are unlikely to benefit from any endocrine therapy.

To assess expression of at least 5, 25, 50, 100 or 200 reporter (ER-related) genes in a sample, the inventors first developed a gene-expression-based ER associated index. ER-positive and ER-negative reference signatures, or centroids, were then described as the median log-transformed expression value of each of the 200 reporter genes in the 209 ER-positive and 77 ER-negative subjects, respectively. For new samples, the similarity between the log-transformed 200-gene ER associated gene expression signature with the reference centroids was determined based on Hoeffding's D statistic (Hollander and Wolfe, 1999). D takes into account the joint rankings of the two variables and thus provides a robust measure of association that, unlike correlation-based statistics, will detect nonmonotonic associations (in statistical terms, it detects a much broader class of alternatives to independence than correlation-based statistics). The ER reporter index (RI) was defined as the difference between the similarities with the ER+ and ER− reference centroids: RI=D⁺−D⁻.

The 200-gene signature of a tumor with high ER-dependent transcriptional activity will resemble more closely the ER-positive centroid and therefore D⁺ will be greater than D⁻ and RI will be positive. The opposite will be the case for tumors with low ER-related activity and thus RI will be small or negative. Subtraction of D⁻ normalizes the reporter index relative to the basal levels of expression of the ER-related genes in ER negative tumors. Because of this and since D is a distribution-free statistic, RI is relatively insensitive to the method used to normalize the microarray data and therefore can be computed across datasets. From the RI, a genomic index of sensitivity to endocrine therapy (SET) was calculated as follows: SET=100(RI+0.2)³. The offset translated RI to mostly positive values and was then transformed to normality using an unconditional Box-Cox power transformation. Finally, the maximum likelihood estimate of the exponent was rounded to the closest integer and the index was scaled to a maximum value of 10.

Embodiments of the present invention also provide a clinically relevant measurement of estrogen receptor (ER) activity within cells by accurately quantifying the transcriptional output due to estrogen receptor activity. This measure or index of the ER pathway or ER activity is an index or measure of the dependence on this growth pathway, and therefore, likely susceptibility to an anti-estrogen receptor hormonal therapy. There are a growing number of hormonal therapies that are used for patients with cancer or to protect from cancer and that vary in their efficacy, cost, and side effects. Aspects of the invention will assist doctors to make improved recommendations about whether and how long to use hormonal therapy for patients with breast cancer or ER-positive breast cancer, particularly those with ER-positive status as established by the existing immunochemical assay, and which hormonal therapy to prescribe for a patient based on the amount of ER-related transcriptional activity measured from a patient's biopsy that indicates the likely sensitivity to hormonal therapy and so matches the treatment selected to the predicted sensitivity to treatment.

Embodiments of the invention are pathway-specific, are applicable to any sample cohort, and are not dependent on inherent biostatistical bias that can limit the accuracy of predictive profiles derived empirically from discovery and validation trial designs linking genes to observed clinical or pathological responses. One advantage of the assay, in addition to its ability to link genomic activity to clinical or pathological response, is that it is quantitative, accurate, and directly comparable using results from different laboratories.

In one aspect of the invention, a calculated index is used to measure the expression of many genes that represent activity of the estrogen receptor pathway within the cells that provides independently predictive information about likely response to hormonal therapy, and that improves the response prediction otherwise obtained by measuring expression of the estrogen receptor alone. The invention includes the methods for standardizing the expression values of future samples to a normalization standard that will allow direct comparison of the results to past samples, such as from a clinical trial. The invention also includes the biostatistical methods to calculate and report the results.

In certain aspects of the invention, measurements of ER and ER-related genes from microarrays have demonstrated to be comparable in standardized datasets from two different laboratories that analyzed two different types of clinical samples (fine needle aspiration cytology samples and surgical tissue samples) and that these accurately diagnose ER status as defined by existing immunochemical assays. In further aspects of the invention, measurements of ER and ER-related genes using this technique have been demonstrated to independently predict distant relapse-free survival in patients who were treated with local therapy (surgery/radiation) followed by post-operative hormonal therapy with tamoxifen. In still further aspects, these gene expression measurements were demonstrated to outperform existing measurements of ER for prediction of survival with this hormonal therapy. In yet still further aspects, measurement of ER-related genes were demonstrated to add to the predictive accuracy of measurements of ER gene expression in the survival analysis of tamoxifen-treated women.

Further embodiments of the invention include kits for the measurement, analysis, and reporting of ER expression and transcriptional output. A kit may include, but is not limited to microarray, quantitative RT-PCR, or other genomic platform reagents and materials, as well as hardware and/or software for performing at least a portion of the methods described. For example, custom microarrays or analysis methods for existing microarrays are contemplated. Also, methods of the invention include methods of accessing and using a reporting system that compares a single result to a scale of clinical trial results. In yet still further aspects of the invention, a digital standard for data normalization is contemplated so that the assay result values from future samples would be able to be directly compared with the assay value results from past samples, such as from specific clinical trials.

The clinical relevance for measurements of ER mRNA and ER related genes from microarrays is also demonstrated herein. Some exemplary advantages to the current composition and methods include, but are not limited to: (1) standardized, quantitative reporting of ER mRNA expression that is comparable in different sample types and laboratories, (2) use of different methods for defining genomic profiles to predict response to adjuvant endocrine treatments, and (3) combining ER-related reporter genes expression to develop a measurable scale or index of estrogen dependence and likely sensitivity to endocrine therapy.

The performance of certain embodiments of a microarray-based ER determination is presented in relation to the current immunohistochemical “gold” standard for evaluation of ER. It is important to remember that IHC assays for ER in routine clinical use are imperfect. The existing IHC assay for ER has only modest positive predictive value (30-60%) for response to various single agent hormonal therapies (Bonneterre et al, 2000; Mouridsen et al, 2001). There are also occasional false negative results. Much of the recognized inter-laboratory differences that affect the IHC results for ER are caused in part by problems associated with tissue fixation methods and antigen retrieval in paraffin tissue sections (Rhodes et al., 2000; Rudiger et al., 2002; Rhodes, 2003; Taylor et al., 1994; Regitnig et al., 2002). Finally, IHC is at least a qualitative assay (reported as positive or negative) and at most a semiquantitative assay (reported as a score). There is still a need to further improve the accuracy with which pathologic assays for ER can predict response to endocrine therapies.

The microarrays provide a suitable method to measure ER expression from clinical samples. ER mRNA levels measured by microarrays, such as Affymetrix U133A gene chips, in fine needle aspirates (FNA), core needle biopsy, and/or frozen tumor tissue samples of breast cancer correlated closely with protein expression by enzyme immunoassay and by routine immunohistochemistry. This is consistent with the previously observed correlation between ER mRNA expression using Northern blot and ER protein expression (Lacroix et al., 2001). An expression level of ER mRNA (ESR1 probe set 205225_)≧500 correctly identified ER-positive tumors (IHC≧10%) with overall accuracy of 96% (95% CI, 90%-99%) in the original set of 82 FNAs and this threshold was validated with 95% overall accuracy (95% CI, 88%-98%) in an independent set of 94 tissue samples (see Table 3). If any ER staining is considered to be ER-positive, the overall accuracy was 98% for FNAs and 99% for tissues. These results indicate that ER status can be reliably determined from gene expression microarray data, with the advantage of providing comparable results from cytologic and surgical samples, and from different laboratories. With appropriately standardized methods for analysis of data, a microarray platform may also provide robust clinical information of ER status.

ER-positive breast cancer includes a continuum of ER expression that might reflect a continuum of biologic behavior and endocrine sensitivity. Others have reported that some breast cancers are difficult to predict as ER-positive based on transcriptional profile and described non-estrogenic growth effects, such as HER-2, more frequently in this small subset of tumors with aggressive natural history (Kun et al., 2003). Indeed, ER mRNA levels are lower in breast cancers that are positive for both ER and HER2 (Konecny et al., 2003). Another group defined a gene expression signature from cDNA arrays that could predict ER protein levels (enzyme immunoassay) and another signature that predicted flow cytometric S-phase measurements (Gruvberger et al., 2004). Their finding of a reciprocal relationship supports the concept that less ER-positive breast cancers are more proliferative. This relationship is also factored into the calculation of the Recurrence Score that adds the values for proliferation and HER-2 gene groups and subtracts the values for the ER gene group (Paik et al., 2004; Paik et al., 2005). Molecular classification from unsupervised cluster analysis shows the same thing by identifying subtypes of luminal-type (ER-positive) breast cancer (Sorlie et al., 2001). The inverse relationship between ER expression and genes associated with proliferation and other growth pathways is best explained by viewing differentiation as a continuum in which cells become increasingly less proliferative and more dependent on ER stimulation as they differentiate. It follows that there would be an inverse relationship between greater sensitivity to endocrine therapy in differentiated tumors and greater sensitivity to chemotherapy in less differentiated tumors. Measurements along this scale could be valuable for treatment selection.

Randomized clinical trials have demonstrated a survival benefit for some patients who receive additional endocrine therapy with an aromatase inhibitor (compared to placebo) after 5 years of adjuvant tamoxifen (Goss et al., 2003; Bryant and Wolmark, 2003). Although there was a 24% relative reduction in deaths after 2.4 years of letrozole, the absolute difference in recurrence or new primaries was only 2.2% at 2.4 years (Goss et al., 2003, Burnstein, 2003). Without a test to identify patients who actually benefit from prolonged adjuvant endocrine therapy, the resulting decision to provide routine extension of adjuvant endocrine treatment (possibly for an indefinite period) in all women with ER-positive cancer could be a costly and potentially avoidable practice for the healthcare community that would benefit an unidentified minority (Buzdar, 2001). It is therefore helpful to consider that this genomic SET index of ER-associated gene expression might identify patients with intermediate endocrine sensitivity as candidates for extended adjuvant endocrine therapy.

A genomic scale of intrinsic endocrine sensitivity might also provide an improved scientific basis for selection of the most appropriate subjects for inclusion in clinical trials. The ATAC and BIG 1-98 trials enrolled 9,366 and 8,010 postmenopausal women, respectively, and both demonstrated 3% absolute improvement in disease-free survival (DFS) at 5 years from adjuvant aromatase inhibition, compared to tamoxifen (Howell et al., 2005; Thurlimann et al., 2005). Aromatase inhibition as first-line endocrine treatment for all postmenopausal women with ER-positive breast cancer would achieve this survival benefit in 3% of patients at significant cost, and might relegate an effective and less expensive treatment (tamoxifen) to relative obscurity. It is also likely that identification of potentially informative subjects, based on predicted partial endocrine sensitivity from indicators such as the SET index, could reduce the size and cost of adjuvant trials, demonstrate larger absolute survival benefit from improved treatment, and establish who should receive each treatment in routine practice after a positive trial result.

As the cost and complexity of endocrine therapy increase, diagnostic tools are needed not merely for prognosis, but, using strong biological rationale, to demonstrate clinical benefit when they are used to guide the selection and duration of endocrine agents therapy. Indicators such as the SET index can predict response to tamoxifen rather than intrinsic prognosis, and should be independent of stage, grade, and the expression levels of ESR1 and PGR. Continuing validation of the SET index with samples from trials of other hormonal agents would help continual refinement of this clinical interpretation.

TABLE 1
Reporter genes for ER-related genomic activity
and use in calculating index

| | | Unigene | Gene | | |
|---|---|---|---|---|---|
| Rank | Probe Set ID | ID | Symbol | Rs | Pg (200) |
| 1 | 209603_at | 169946 | GATA3 | 0.783 | 1.000 |
| 2 | 215304_at | 159264 | | 0.779 | 1.000 |
| 3 | 218195_at | 15929 | C6orf211 | 0.774 | 1.000 |
| 4 | 212956_at | 411317 | KIAA0882 | 0.771 | 1.000 |
| 5 | 209604_s_at | 169946 | GATA3 | 0.764 | 1.000 |
| 6 | 202088_at | 79136 | SLC39A6 | 0.757 | 1.000 |
| 7 | 209602_s_at | 169946 | GATA3 | 0.749 | 1.000 |
| 8 | 212496_s_at | 301011 | JMJD2B | 0.733 | 1.000 |
| 9 | 212960_at | 411317 | KIAA0882 | 0.724 | 1.000 |
| 10 | 215867_x_at | 5344 | AP1G1 | 0.724 | 1.000 |
| 11 | 214164_x_at | 512620 | CA12 | 0.721 | 1.000 |
| 12 | 203963_at | 512620 | CA12 | 0.719 | 1.000 |
| 13 | 41660_at | 252387 | CELSR1 | 0.709 | 1.000 |
| 14 | 218259_at | 151076 | MRTF-B | 0.695 | 1.000 |
| 15 | 204667_at | 163484 | FOXA1 | 0.689 | 1.000 |
| 16 | 211712_s_at | 430324 | ANXA9 | 0.684 | 1.000 |
| 17 | 218532_s_at | 82273 | FLJ201S2 | 0.677 | 1.000 |
| 18 | 212970_at | 15740 | FLJ14001 | 0.677 | 1.000 |
| 19 | 209459_s_at | 1588 | ABAT | 0.676 | 0.999 |
| 20 | 204508_s_at | 512620 | CA12 | 0.675 | 1.000 |
| 21 | 218976_at | 260720 | DNAJC12 | 0.673 | 0.998 |
| 22 | 217838_s_at | 241471 | EVL | 0.673 | 1.000 |
| 23 | 218211_s_at | 297405 | MLPH | 0.669 | 1.000 |
| 24 | 222275_at | 124165 | MRPS30 | 0.666 | 1.000 |
| 25 | 218471_s_at | 129213 | BBS1 | 0.666 | 0.999 |
| 26 | 214053_at | 7888 | | 0.666 | 0.999 |
| 27 | 203438_at | 155223 | STC2 | 0.664 | 1.000 |
| 28 | 213234_at | 6189 | KIAA1467 | 0.664 | 0.999 |
| 29 | 219197_s_at | 435861 | SCUBE2 | 0.657 | 0.999 |
| 30 | 212692_s_at | 209846 | LRBA | 0.657 | 0.999 |
| 31 | 200711_s_at | 171626 | SKP1A | 0.654 | 1.000 |
| 32 | 205074_at | 15813 | SLC22A5 | 0.653 | 1.000 |
| 33 | 203685_at | 501181 | BCL2 | 0.653 | 1.000 |
| 34 | 209460_at | 1588 | ABAT | 0.653 | 0.999 |
| 35 | 222125_s_at | 271224 | PH-4 | 0.651 | 1.000 |
| 36 | 204798_at | 407830 | MYB | 0.651 | 0.999 |
| 37 | 212985_at | 15740 | FLJ14001 | 0.648 | 1.000 |
| 38 | 203929_s_at | 101174 | MAPT | 0.647 | 0.998 |
| 39 | 202089_s_at | 79136 | SLC39A6 | 0.642 | 0.997 |
| 40 | 205696_s_at | 444372 | GFRA1 | 0.639 | 0.997 |
| 41 | 209681_at | 30246 | SLC19A2 | 0.637 | 0.999 |
| 42 | 212495_at | 301011 | JMJD2B | 0.637 | 0.999 |
| 43 | 218510_x_at | 82273 | FLJ20152 | 0.634 | 0.995 |
| 44 | 208682_s_at | 376719 | MAGED2 | 0.632 | 0.994 |
| 45 | 212195_at | 529772 | | 0.630 | 0.997 |
| 46 | 51192_at | 29173 | SSH-3 | 0.630 | 0.999 |
| 47 | 40016_g_at | 212787 | KIAA0303 | 0.628 | 0.997 |
| 48 | 212638_s_at | 450060 | WWP1 | 0.627 | 0.994 |
| 49 | 218692_at | 354793 | FLJ20366 | 0.624 | 0.991 |
| 50 | 213077_at | 283283 | FLJ21940 | 0.623 | 0.985 |
| 51 | 203439_s_at | 155223 | STC2 | 0.623 | 0.995 |
| 52 | 212441_at | 79276 | KIAA0232 | 0.622 | 0.988 |
| 53 | 210652_s_at | 112949 | C1orf34 | 0.621 | 0.990 |
| 54 | 219981_x_at | 288995 | ZNF587 | 0.620 | 0.984 |
| 55 | 205186_at | 406050 | DNALI1 | 0.620 | 0.990 |
| 56 | 213627_at | 376719 | MAGED2 | 0.620 | 0.987 |
| 57 | 200670_at | 437638 | XBP1 | 0.617 | 0.985 |
| 58 | 218437_s_at | 30824 | LZTFL1 | 0.617 | 0.987 |
| 59 | 206754_s_at | 1360 | CYP2B6 | 0.616 | 0.985 |
| 60 | 209696_at | 360509 | FBP1 | 0.616 | 0.987 |
| 61 | 201826_s_at | 238126 | CGI-49 | 0.615 | 0.984 |
| 62 | 219833_s_at | 446047 | EFHC1 | 0.610 | 0.975 |
| 63 | 203928_x_at | 101174 | MAPT | 0.610 | 0.976 |
| 64 | 216092_s_at | 22891 | SLC7A8 | 0.609 | 0.985 |
| 65 | 200810_s_at | 437351 | CIRBP | 0.609 | 0.977 |
| 66 | 204811_s_at | 389415 | CACNA2D2 | 0.609 | 0.968 |
| 67 | 44654_at | 294005 | G6PC3 | 0.609 | 0.974 |
| 68 | 202371_at | 194329 | FLJ21174 | 0.608 | 0.970 |
| 69 | 209173_at | 226391 | AGR2 | 0.607 | 0.971 |
| 70 | 212196_at | 529772 | | 0.606 | 0.953 |
| 71 | 210720_s_at | 324104 | APBA2BP | 0.606 | 0.965 |
| 72 | 204497_at | 20196 | ADCY9 | 0.605 | 0.965 |
| 73 | 214440_at | 155956 | NAT1 | 0.604 | 0.960 |
| 74 | 205009_at | 350470 | TFF1 | 0.603 | 0.964 |
| 75 | 204862_s_at | 81687 | NME3 | 0.601 | 0.971 |
| 76 | 219562_at | 3797 | RAB26 | 0.600 | 0.949 |
| 77 | 50965_at | 3797 | RAB26 | 0.599 | 0.951 |
| 78 | 218966_at | 111782 | MYO5C | 0.598 | 0.961 |
| 79 | 217979_at | 364544 | TM4SF13 | 0.596 | 0.972 |
| 80 | 209759_s_at | 403436 | DCI | 0.596 | 0.938 |
| 81 | 212637_s_at | 450060 | WWP1 | 0.594 | 0.951 |
| 82 | 218094_s_at | 256086 | C20orf35 | 0.592 | 0.954 |
| 83 | 219222_at | 11916 | RBKS | 0.592 | 0.941 |
| 84 | 202121_s_at | 12107 | BC-2 | 0.591 | 0.940 |
| 85 | 215001_s_at | 442669 | GLUL | 0.591 | 0.940 |
| 86 | 210085_s_at | 430324 | ANXA9 | 0.590 | 0.934 |
| 87 | 210958_s_at | 212787 | KIAA0303 | 0.589 | 0.940 |
| 88 | 201596_x_at | 406013 | KRT18 | 0.588 | 0.928 |
| 89 | 212209_at | 435249 | THRAP2 | 0.587 | 0.923 |
| 90 | 221139_s_at | 279815 | CSAD | 0.586 | 0.924 |
| 91 | 201384_s_at | 458271 | M17S2 | 0.586 | 0.910 |
| 92 | 213283_s_at | 416358 | SALL2 | 0.586 | 0.927 |
| 93 | 202908 at | 26077 | WFS1 | 0.585 | 0.917 |
| 94 | 219786_at | 121378 | MTL5 | 0.585 | 0.918 |
| 95 | 214109_at | 209846 | LRBA | 0.584 | 0.930 |
| 96 | 203791_at | 181042 | DMXL1 | 0.583 | 0.914 |
| 97 | 205012_s_at | 155482 | HAGH | 0.583 | 0.903 |
| 98 | 212492_s_at | 301011 | JMJD2B | 0.582 | 0.902 |
| 99 | 218026_at | 16059 | HSPC009 | 0.579 | 0.905 |
| 100 | 210272_at | 1360 | CYP2B6 | 0.579 | 0.897 |
| 101 | 204199_at | 432842 | RALGPS1 | 0.577 | 0.892 |
| 102 | 202752_x_at | 22891 | SLC7A8 | 0.577 | 0.886 |
| 103 | 217645_at | 531103 | | 0.576 | 0.882 |
| 104 | 213419_at | 324125 | APBB2 | 0.576 | 0.888 |
| 105 | 219919_s_at | 29173 | SSH-3 | 0.575 | 0.861 |
| 106 | 213365_at | 248437 | MGC16943 | 0.574 | 0.861 |
| 107 | 219206_x_at | 126372 | CGI-119 | 0.574 | 0.883 |
| 108 | 221751_at | 388400 | PANK3 | 0.573 | 0.875 |
| 109 | 211596_s_at | 528353 | LRIG1 | 0.572 | 0.863 |
| 110 | 221963_x_at | 356530 | | 0.572 | 0.867 |
| 111 | 202641_at | 182215 | ARL3 | 0.572 | 0.850 |
| 112 | 201754_at | 351875 | COX6C | 0.571 | 0.857 |
| 113 | 219741_x_at | 515644 | ZNF552 | 0.569 | 0.848 |
| 114 | 209224_s_at | | NDUFA2 | 0.568 | 0.862 |
| 115 | 212099_at | 406064 | RHOB | 0.568 | 0.836 |
| 116 | 205794_s_at | 292511 | NOVA1 | 0.568 | 0.836 |
| 117 | 219913_s_at | 171342 | CRNKL1 | 0.568 | 0.816 |
| 118 | 204934_s_at | 432750 | HPN | 0.567 | 0.830 |
| 119 | 209341_s_at | 413513 | IKBKB | 0.567 | 0.816 |
| 120 | 204231_s_at | 528334 | FAAH | 0.567 | 0.817 |
| 121 | 203571_s_at | 511763 | C10orf116 | 0.567 | 0.807 |
| 122 | 204045_at | 95243 | TCEAL1 | 0.566 | 0.833 |
| 123 | 202636_at | 147159 | RNF103 | 0.566 | 0.788 |
| 124 | 202962_at | 15711 | KIF13B | 0.565 | 0.798 |
| 125 | 208865_at | 318381 | CSNK1A1 | 0.563 | 0.801 |
| 126 | 201825_s_at | 238126 | CGI-49 | 0.563 | 0.806 |
| 127 | 219686_at | 58241 | STK32B | 0.562 | 0.806 |
| 128 | 57540_at | 11916 | RBKS | 0.560 | 0.782 |
| 129 | 212416_at | 31218 | SCAMP1 | 0.559 | 0.801 |
| 130 | 201170_s_at | 171825 | BHLHB2 | 0.559 | 0.758 |
| 131 | 40093_at | 155048 | LU | 0.558 | 0.773 |
| 132 | 219414_at | 12079 | CLSTN2 | 0.557 | 0.761 |
| 133 | 209623_at | 167531 | MCCC2 | 0.556 | 0.758 |
| 134 | 202772_at | 444925 | HMGCL | 0.555 | 0.752 |
| 135 | 208517_x_at | 446567 | BTF3 | 0.553 | 0.734 |
| 136 | 213018_at | 21145 | ODAG | 0.552 | 0.764 |
| 137 | 204703_at | 251328 | TTC10 | 0.551 | 0.731 |
| 138 | 203801_at | 247324 | MRPS14 | 0.551 | 0.730 |
| 139 | 203246_s_at | 437083 | TUSC4 | 0.550 | 0.733 |
| 140 | 218769_s_at | 239154 | ANKRA2 | 0.549 | 0.740 |
| 141 | 203476_at | 82128 | TPBG | 0.549 | 0.706 |
| 142 | 217770_at | 437388 | PIGT | 0.548 | 0.736 |
| 143 | 35666_at | 32981 | SEMA3F | 0.547 | 0.694 |
| 144 | 212508_at | 24719 | MOAP1 | 0.546 | 0.686 |
| 145 | 208712_at | 371468 | CCND1 | 0.545 | 0.703 |
| 146 | 204863_s_at | 71968 | IL6ST | 0.544 | 0.710 |
| 147 | 204284_at | 303090 | PPP1R3C | 0.544 | 0.672 |
| 148 | 203628_at | 239176 | IGF1R | 0.544 | 0.674 |
| 149 | 200719_at | 171626 | SKP1A | 0.544 | 0.668 |
| 150 | 214919_s_at | | MASK-BP3 | 0.544 | 0.669 |
| 151 | 205376_at | 153687 | INPP4B | 0.544 | 0.691 |
| 152 | 202263_at | 334832 | CYB5R1 | 0.543 | 0.674 |
| 153 | 218450_at | 294133 | HEBP1 | 0.543 | 0.660 |
| 154 | 213285_at | 146180 | LOC161291 | 0.543 | 0.666 |
| 155 | 209740_s_at | 264 | DXS1283E | 0.543 | 0.653 |
| 156 | 205380_at | 15456 | PDZK1 | 0.543 | 0.661 |
| 157 | 203144_s_at | 368916 | KIAA0040 | 0.543 | 0.656 |
| 158 | 214552_s_at | 390163 | RABEP1 | 0.542 | 0.660 |
| 159 | 202814_s_at | 15299 | HIS1 | 0.540 | 0.629 |
| 160 | 205776_at | 396595 | FMO5 | 0.539 | 0.633 |
| 161 | 217906_at | 415236 | KLHDC2 | 0.539 | 0.640 |
| 162 | 212148_at | 408222 | PBX1 | 0.539 | 0.620 |
| 163 | 220581_at | 287738 | C6orf97 | 0.538 | 0.643 |
| 164 | 200811_at | 437351 | CIRBP | 0.538 | 0.574 |
| 165 | 217894_at | 239155 | KCTD3 | 0.538 | 0.580 |
| 166 | 206197_at | 72050 | NME5 | 0.537 | 0.610 |
| 167 | 202454_s_at | 306251 | ERBB3 | 0.537 | 0.614 |
| 168 | 218394_at | 22795 | FLJ22386 | 0.535 | 0.601 |
| 169 | 201413_at | 356894 | HSD17B4 | 0.535 | 0.593 |
| 170 | 40569_at | 458361 | ZNF42 | 0.535 | 0.574 |
| 171 | 221856_s_at | 3346 | FLJ11280 | 0.535 | 0.576 |
| 172 | 210336_x_at | 458361 | ZNF42 | 0.534 | 0.584 |
| 173 | 211621_at | 99915 | AR | 0.533 | 0.573 |
| 174 | 204623_at | 82961 | TFF3 | 0.533 | 0.533 |
| 175 | 40148_at | 324125 | APBB2 | 0.533 | 0.581 |
| 176 | 212446_s_at | 387400 | LASS6 | 0.532 | 0.543 |
| 177 | 210735_s_at | 279916 | CA12 | 0.531 | 0.540 |
| 178 | 214924_s_at | 457063 | OIP106 | 0.531 | 0.561 |
| 179 | 203071_at | 82222 | SEMA3B | 0.531 | 0.522 |
| 180 | 213527_s_at | 301463 | LOC146542 | 0.530 | 0.531 |
| 181 | 208617_s_at | 82911 | PTP4A2 | 0.530 | 0.517 |
| 182 | 213249_at | 76798 | FBXL7 | 0.529 | 0.552 |
| 183 | 205645_at | 334168 | REPS2 | 0.529 | 0.520 |
| 184 | 208788_at | 343667 | ELOVL5 | 0.529 | 0.543 |
| 185 | 205769_at | 11729 | SLC27A2 | 0.528 | 0.501 |
| 186 | 213712_at | 246107 | ELOVL2 | 0.528 | 0.510 |
| 187 | 212697_at | 432850 | LOC162427 | 0.528 | 0.503 |
| 188 | 219900_s_at | 435303 | FLJ20626 | 0.528 | 0.485 |
| 189 | 213832_at | 23729 | | 0.527 | 0.490 |
| 190 | 213049_at | 167031 | GARNL1 | 0.527 | 0.474 |
| 191 | 59437_at | 414028 | C9orf116 | 0.527 | 0.504 |
| 192 | 204072_s_at | 390874 | 13CDNA73 | 0.526 | 0.451 |
| 193 | 210108_at | 399966 | CACNA1D | 0.526 | 0.489 |
| 194 | 214855_s_at | 167031 | GARNL1 | 0.525 | 0.459 |
| 195 | 209662_at | 528302 | CETN3 | 0.525 | 0.441 |
| 196 | 219687_at | 58650 | MART2 | 0.525 | 0.470 |
| 197 | 217191_x_at | | COX6CP1 | 0.524 | 0.440 |
| 198 | 203538_at | 13572 | CAMLG | 0.524 | 0.442 |
| 199 | 213702_x_at | 324808 | ASAH1 | 0.522 | 0.456 |
| 200 | 212744_at | 26471 | BBS4 | 0.522 | 0.458 |

In some aspects, although not intending to bound to any single theory, the ER reporter index can be of importance for tumors with high ER mRNA expression. If ER mRNA and the reporter index are high, this can describe a highly endocrine-dependent state for which tamoxifen alone seems to be sufficient for prolonged survival benefit. Patients with high ER mRNA expression but low reporter index appear to derive initial benefit from tamoxifen, but that is not sustained over the long term. Those patients' tumors are likely to be partially endocrine-dependent and might benefit from more potent endocrine therapy in the adjuvant setting. Some women might also benefit from more potent endocrine therapy. A measurable scale of ER gene expression and genomic activity might be applicable to any endocrine therapy that targets ER or other hormonal receptor activity. The relation of an index to efficacy of different endocrine therapies could be used to guide the selection of first-line treatment (e.g., chemotherapy versus endocrine therapy), influence the selection of endocrine agent based on likely endocrine sensitivity, and possibly to re-evaluate endocrine sensitivity if ER-positive breast cancer recurs.

Typically for clinical utility one would define the optimal probe set for ESR1 (ERα gene) on the Affymetrix U133A GeneChip™ to measure ER gene expression. The ESR1 205225_probe set produces the highest median and greatest range of expression and the strongest correlation with ER status because this probe set recognizes the most 3′ end of ESR1 (NetAffx search tool at www.affymetrix.com). The initial reverse transcription (RT) of mRNA sequences in each sample begins at the unique poly-A tail at the 3′ end of mRNA. Therefore, the 3′ end is likely to be the most represented part of any mRNA sequence, and probes that target the 3′ end generally produce the strongest hybridization signal.

In other aspects of the invention it is preferred that biostatistical methods be used that allow standardization of microarray data from any contributing laboratory. At present, direct comparison of IHC results for ER from multiple centers is difficult because technical staining methods differ, positive and negative tissue controls are laboratory-dependent, and interpretation of staining is subjective to the interpretation of the individual pathologist or the threshold setting of the image analysis system being used (Rhodes et al., 2000; Rhodes, 2003; Regitnig et al., 2002). Even in quantitative RT-PCR assays, the expression of genes of interest are calculated relative to only one or several intrinsic housekeeper genes in each assay. The techniques for RNA extraction from fresh samples and preparation for hybridization to Affymetrix microarrays are available from standardized laboratory protocols. However, it should not be overlooked that uniform normalization of microarray data from every breast cancer sample to a digital standard (e.g., U133A dCHIP dataset) will consistently calculate the expression of all genes of interest relative to the expression of thousands of intrinsic control genes. This availability of multiple controls to standardize expression levels of all genes on the microarray is a robust mathematical control that can explain the comparable results from measurements of ER mRNA expression levels in different sample types and in different laboratories. Adoption of an agreed dCHIP standard for data normalization of breast cancer samples using the Affymetrix U133A array could lead to a digital standard available to laboratories for clinical trials and for routine diagnostics.

The implications of establishing standard analysis tools for development of a useful clinical assay are clear. When diagnostic microarrays are introduced into the clinic through a central reference laboratory, then uniform data normalization and standardized experimental procedure require internal quality control procedures by the central laboratory. However, in a decentralized system where each center performs its own profiling following a standard procedure using the same microarray platform, a single digital standard should be available for data normalization. This allows different laboratories to generate data that is directly comparable to a common standard.

TABLE 2
Genes indicative of the responsiveness of a cancer cell to therapy

| Probe.Set | Accession | Name | T-stat | P-val |
|---|---|---|---|---|
| 203930_s_at | NM_016835.1 | Microtubule-associated protein | −6.42 | 5.25 × 10-08 |
| 212745_s_at | A1813772 | Bardet-Biedl syndrome 4 | −6.25 | 9.40 × 10-08 |
| 203928_x_at | NM_016835.1 | Microtubule-associated protein | −5.99 | 2.70 × 10-07 |
| 206401_s_at | J03778.1 | Microtubule-associated protein | −5.73 | 7.02 × 10-07 |
| 203929_s_at | NM_016835.1 | Microtubule-associated protein | −5.52 | 1.26 × 10-06 |
| 212207_at | AK023837.1 | KIAA1025 protein | −5.37 | 2.21 × 10-06 |
| 212046_x_at | X60188.1 | Mitogen-activated protein kinase | −5.33 | 3.43 × 10-06 |
| 210469_at | BC002915.1 | Discs, large (Drosophila) homol | −5.28 | 3.53 × 10-06 |
| 205074_at | NM_003060.1 | Solute carrier family 22 (organ | −5.13 | 5.45 × 10-06 |
| 204509_at | NM_017689.1 | Hypothetical protein FLJ20151 | −5.02 | 6.15 × 10-06 |
| 205696_s_at | NM_005264.1 | GDNF family receptor alpha 1 | −5.00 | 1.06 × 10-05 |
| 219741_x_at | NM_024762.1 | Hypothetical protein FLJ21603 | −4.94 | 1.00 × 10-05 |
| 215616_s_at | AB020683.1 | KIAA0876 protein | −4.86 | 1.43 × 10-05 |
| 208945_s_at | NM_003766.1 | Beclin 1 (coiled-coil, myosin-l | −4.86 | 1.48 × 10-05 |
| 217542_at | BE930512 | ESTs | −4.80 | 1.84 × 10-05 |
| 202204_s_at | AF124145.1 | Autocrine motility factor recep | −4.74 | 2.05 × 10-05 |
| 204916_at | NM_005855.1 | Receptor (calcitonin) activity | −4.70 | 2.92 × 10-05 |
| 218769_s_at | NM_023039.1 | Ankyrin repeat, family A (RFXAN | −4.70 | 2.58 × 10-05 |
| 219981_x_at | NM_017961.1 | Hypothetical protein FLJ20813 | −4.66 | 4.44 × 10-05 |
| 222131_x_at | BC004327.1 | Hypothetical protein BC014942 | −4.64 | 3.26 × 10-05 |
| 213234_at | AB040900.1 | KIAA1467 protein | −4.60 | 3.73 × 10-05 |
| 219197_s_at | AI424243 | CEGP1 protein | −4.57 | 3.45 × 10-05 |
| 205425_at | NM_005338.3 | Huntington interacting protein | −4.51 | 8.86 × 10-05 |
| 213504_at | W63732 | COP9 subunit 6 (MOV34 homolog, | −4.50 | 4.98 × 10-05 |
| 201413_at | NM_000414.1 | Hydroxysteroid (17-beta) dehydr | −4.46 | 5.71 × 10-05 |
| 203050_at | NM_005657.1 | Tumor protein p53 binding prote | −4.45 | 7.53 × 10-05 |
| 212494_at | AB028998.1 | KIAA1075 protein | −4.43 | 9.46 × 10-05 |
| 209173_at | AF088867.1 | Anterior gradient 2 homolog (Xe | −4.41 | 6.36 × 10-05 |
| 201124_at | AL048423 | Integrin, beta 5 | −4.41 | 7.76 × 10-O5 |
| 205354_at | NM_000156.3 | Guanidinoacetate N-methyltransf | −4.39 | 8.11 × 10-05 |
| 212444_at | AA156240 | Homo sapiens cDNA: FLJ22182 fis | −4.37 | 7.71 × 10-05 |
| 205225_at | NM_000125.1 | Estrogen receptor 1 | −4.37 | 8.12 × 10-05 |
| 211000_s_at | AB015706.1 | Interleukin 6 signal transducer | −4.36 | 9.16 × 10-05 |
| 204012_s_at | AL529189 | KIAA0547 gene product | −4.36 | 8.63 × 10-05 |
| 203682_s_at | NM_002225.2 | Isovaleryl Coenzyme A dehydroge | −4.35 | 7.60 × 10-05 |
| 220357_s_at | NM_016276.1 | Serum/glucocorticoid regulated | −4.35 | 5.94 × 10-05 |
| 216173_at | AK025360.1 | Homo sapiens cDNA: FLJ21707 fis | −4.32 | 7.65 × 10-05 |
| 210230_at | BC003629.1 | RNA, U2 small nuclear | −4.26 | 9.95 × 10-05 |
| 219044_at | NM_018271.1 | Hypothetical protein FLJ10916 | −4.25 | 1.75 × 10-04 |
| 218761_at | NM_017610.1 | Likely ortholog of mouse Arkadi | −4.23 | 1.35 × 10-04 |
| 210826_x_at | AF098533.1 | RAD17 homolog (S. pombe) | −4.22 | 1.44 × 10-04 |
| 210831_s_at | L27489.1 | Prostaglandin E receptor 3 (sub | −4.22 | 1.07 × 10-04 |
| 211233_x_at | M12674.1 | Estrogen receptor 1 | −4.21 | 1.20 × 10-04 |
| 218807_at | NM_006113.2 | Vav 3 oncogene | −4.20 | 1.46 × 10-04 |
| 210129_s_at | AF078842.1 | DKFZP434B103 protein | −4.19 | 1.09 × 10-04 |
| 39313_at | AB002342 | Protein kinase, lysine deficien | −4.19 | 1.23 × 10-04 |
| 213245_at | AL120173 | Homosapiens cDNA FLJ30781 fis, | −4.18 | 1.43 × 10-04 |
| 214053_at | AW772192 | Homo sapiens clone 23736 mRNA s | −4.18 | 1.51 × 10-04 |
| 205352_at | NM_005025.1 | Serine (or cysteine) proteinase | −4.17 | 1.47 × 10-04 |
| 213623_at | NM_007054.1 | Kinesin family member 3A | −4.15 | 1.88 × 10-04 |
| 215304_at | U79293.1 | Human clone 23948 mRNA sequence | −4.13 | 1.40 × 10-04 |
| 203009_at | NM_005581.1 | Lutheran blood group (Auberger | −4.13 | 1.80 × 10-04 |
| 218692_at | NM_017786.1 | Hypothetical protein FLJ20366 | −4.13 | 1.76 × 10-04 |
| 218976_at | NM_021800.1 | J domain containing protein 1 | −4.12 | 1.76 × 10-04 |
| 201405_s_at | NM_006833.1 | COP9 subunit 6 (MOV34 homolog, | −4.11 | 1.63 × 10-04 |
| 202168_at | NM_003187.1 | TAF9 RNA polymerase II, TATA bo | −4.11 | 2.01 × 10-04 |
| 216109_at | AK025348.1 | Homo sapiens cDNA: FLJ21695 fis | −4.11 | 1.77 × 10-04 |
| 219051_x_at | NM_024042.1 | Hypothetical protein MGC2601 | −4.10 | 2.34 × 10-04 |
| 210908_s_at | AB055804.1 | Prefoldin 5 | −4.09 | 1.71 × 10-04 |
| 221728_x_at | AK025198.1 | Homo sapiens cDNA FLJ30298 fis, | −4.07 | 2.11 × 10-04 |
| 203187_at | NM_001380.1 | Dedicator of cyto-kinesis 1 | −4.06 | 2.22 × 10-04 |
| 212660_at | AI735639 | KIAA0239 protein | −4.04 | 2.56 × 10-04 |
| 212956_at | AB020689.1 | KIAA0882 protein | −4.01 | 2.27 × 10-04 |
| 217838_s_at | NM_016337.1 | RNB6 | −4.01 | 2.14 × 10-04 |
| 218621_at | NM_016173.1 | HEMK homolog 7 kb | −4.01 | 1.92 × 10-04 |
| 201681_s_at | AB0111855.1 | Discs, large (Drosophila) homol | −4.01 | 2.49 × 10-04 |
| 209884_s_at | AF047033.1 | Solute carrier family 4, sodium | −4.00 | 2.98 × 10-04 |
| 201557_at | NM_014232.1 | Vesicle-associated membrane pro | −3.99 | 2.23 × 10-04 |
| 219338_s_at | NM_017691.1 | Hypothetical protein FLJ20156 | −3.99 | 2.94 × 10-04 |
| 217828_at | NM_024755.1 | Hypothetical protein FLJ13213 | −3.98 | 2.42 × 10-04 |
| 209339_at | U76248.1 | Seven in absentia homolog 2 (Dr | −3.98 | 2.26 × 10-04 |
| 214218_s_at | AV699347 | Homo sapiens cDNA FLJ30298 fis, | −3.97 | 2.82 × 10-04 |
| 221643_s_at | AF016005.1 | Arginine-glutamic acid dipeptid | −3.96 | 2.57 × 10-04 |
| 218211_s_at | NM_024101.1 | Melanophilin | −3.95 | 3.05 × 10-04 |
| 221483_sat | AF084555.1 | Cyclic AMP phosphoprotein, 19 k | −3.95 | 2.83 × 10-04 |
| 211864_s_at | AF207990.1 | Fer-1-like 3, myoferlin (C. ele | −3.92 | 3.29 × 10-04 |
| 202392_s_at | NM_014338.1 | Phosphatidylserine decarboxylas | −3.92 | 4.33 × 10-04 |
| 214164_x_at | BF752277 | Adaptor-related protein complex | −3.91 | 3.52 × 10-04 |
| 204862_s_at | NM_002513.1 | Non-metastatic cells 3, protein | −3.91 | 3.55 × 10-04 |
| 215552_s_at | AI073549 | Estrogen receptor 1 | −3.91 | 3.33 × 10-04 |
| 211235_s_at | AF258450.1 | Estrogen receptor 1 | −3.90 | 3.13 × 10-04 |
| 210833_at | AL031429 | Prostaglandin E receptor 3 (sub | −3.89 | 3.06 × 10-04 |
| 204660_at | NM_005262.1 | Growth factor, augmenter of liv | −3.89 | 2.79 × 10-04 |
| 211234_x_at | AF258449.1 | Estrogen receptor 1 | −3.89 | 3.10 × 10-04 |
| 201508_at | NM_001552.1 | Insulin-like growth factor bind | −3.88 | 4.04 × 10-04 |
| 213527_s_at | AI350500 | Similar to hypothetical protein | −3.85 | 4.33 × 10-04 |
| 202048_s_at | NM_014292.1 | Chromobox homolog 6 | −3.84 | 4.15 × 10-04 |
| 206794_at | NM_005235.1 | v-erb-a erythroblastic leukemia | −3.84 | 3.87 × 10-04 |
| 201798_s_at | NM_013451.1 | Fer-1-like 3, myoferlin (C. ele | −3.83 | 4.44 × 10-04 |
| 213523_at | AI671049 | Cyclin E1 | 3.81 | 4.14 × 10-04 |
| 209050_s_at | AI421559 | Ral guanine nucleotide dissocia | 3.83 | 4.07 × 10-04 |
| 217294_s_at | U88968.1 | Enolase 1, (alpha) | 3.84 | 4.48 × 10-04 |
| 201555_at | NM_002388.2 | MCM3 minichromosome maintenance | 3.84 | 4.41 × 10-04 |
| 201030_x at | NM_002300.1 | Lactate dehydrogenase B | 3.85 | 3.85 × 10-04 |
| 202912_at | NM_001124.1 | Adrenomedullin | 3.86 | 3.59 × 10-04 |
| 204050_s_at | NM_001833.1 | Clathrin, light polypeptide (Lc | 3.88 | 3.97 × 10-04 |
| 202342_s_at | NM_015271.1 | Tripartite motif-containing 2 | 3.88 | 4.43 × 10-04 |
| 209393_s_at | AF047695.1 | Eukaryotic translation initiati | 3.89 | 4.21 × 10-04 |
| 219774_at | NM_019044.1 | Hypothetical protein FLJ10996 | 3.93 | 3.86 × 10-04 |
| 204162_at | NM_006101.1 | Highly expressed in cancer, nc | 3.93 | 2.94 × 10-04 |
| 216237_s_at | AA807529 | MCM5 minichromosome maintenance | 3.96 | 2.84 × 10-04 |
| 214581_x_at | BE568134 | Tumor necrosis factor receptor | 3.99 | 3.07 × 10-04 |
| 209408_at | U63743.1 | Kinesin-like 6 (mitotic centrom | 3.99 | 2.23 × 10-04 |
| 208370_s_at | NM_004414.2 | Down syndrome critical region g | 4.02 | 2.94 × 10-04 |
| 203744_at | NM_005342.1 | High-mobility group box 3 | 4.02 | 2.02 × 10-04 |
| 209575_at | BC001903.1 | Interleukin 10 receptor, beta | 4.03 | 2.84 × 10-04 |
| 200934_at | NM_003472.1 | DEK oncogene (DNA binding) | 4.05 | 2.54 × 10-04 |
| 202341_s_at | AA149745 | Tripartite motif-containing 2 | 4.06 | 2.87 × 10-04 |
| 200996_at | NM005721.2 | ARP3 actin-related protein 3 ho | 4.06 | 2.42 × 10-04 |
| 206392_s_at | NM_002888.1 | Retinoic acid receptor responde | 4.06 | 2.28 × 10-04 |
| 206391_at | NM_002888.1 | Retinoic acid receptor responde | 4.07 | 2.52 × 10-04 |
| 201797_s_at | NM_006295.1 | Valyl-tRNA synthetase 2 | 4.07 | 2.17 × 10-04 |
| 209358_at | AF118094.1 | TAF11 RNA polymerase II, TATA b | 4.07 | 2.34 × 10-04 |
| 209201_x_at | L01639.1 | Chemokine (C-X-C motif) recepto | 4.09 | 2.80 × 10-04 |
| 209016_s_at | BC002700.1 | Keratin 7 | 4.14 | 1.69 × 10-04 |
| 221957_at | BF939522 | Pyruvate dehydrogenase kinase, | 4.15 | 2.22 × 10-04 |
| 218350_s_at | NM_015895.1 | Geminin, DNA replication inhibi | 4.16 | 1.64 × 10-04 |
| 201897_s_at | NM_001826.1 | p53-regulated DDA3 | 4.21 | 1.36 × 10-04 |
| 209642_at | AF043294.2 | BUB1 budding uninhibited by ben | 4.22 | 1.22 × 10-04 |
| 201930_at | NM_005915.2 | MCM6 minichromosome maintenance | 4.23 | 1.16 × 10-04 |
| 202870_s_at | NM_001255.1 | CDC20 cell division cycle 20 ho | 4.23 | 1.07 × 10-04 |
| 221485_at | NM_004776.1 | UDP-Gal:betaGlcNAc beta 1,4- ga | 4.26 | 1.08 × 10-04 |
| 211919_s_at | AF348491.1 | Chemokine (C-X-C motif) recepto | 4.27 | 1.61 × 10-04 |
| 218887_at | NM_015950.1 | Mitochondrial ribosomal protein | 4.27 | 8.93 × 10-05 |
| 216295_s_at | X81636.1 | H.sapiens clathrin light chain | 4.28 | 1.17 × 10-04 |
| 218726_at | NM_018410.1 | Hypothetical protein DKFZp762E1 | 4.28 | 1.19 × 10-04 |
| 204989_s_at | BF305661 | Integrin, beta 4 | 4.30 | 1.01 × 10-04 |
| 221872_at | AI669229 | Retinoic acid receptor responde | 4.31 | 1.12 × 10-04 |
| 206746_at | NM_001195.2 | Beaded filament structural prot | 4.32 | 9.33 × 10-05 |
| 201231_s_at | NM_001428.1 | Enolase 1, (alpha) | 4.42 | 5.76 × 10-05 |
| 204203_at | NM_001806.1 | CCAAT/enhancer binding protein | 4.42 | 6.44 × 10-05 |
| 211555_s_at | AF020340.1 | Guanylate cyclase 1, soluble, b | 4.47 | 5.11 × 10-05 |
| 202200_s_at | NM_003137.1 | SFRS protein kinase 1 | 4.47 | 5.17 × 10-05 |
| 213101_s_at | Z78330 | Homo sapiens mRNA; cDNA DKFZp68 | 4.49 | 7.76 × 10-05 |
| 204600_at | NM_004443.1 | EphB3 | 4.51 | 5.81 × 10-05 |
| 212689_s_at | AA524505 | Zinc finger protein | 4.52 | 5.10 × 10-05 |
| 209773_s_at | BC001886.1 | Ribonucleotide reductase M2 po1 | 4.55 | 3.18 × 10-05 |
| 204962_s_at | NM_001809.2 | Centromere protein A, l7kDa | 4.62 | 3.00 × 10-05 |
| 211519_s_at | AY026505.1 | Kinesin-like 6 (mitotic centrom | 4.62 | 2.41 × 10-05 |
| 204825_at | NM_014791.1 | Maternal embryonic leucine zipp | 4.73 | 2.45 × 10-05 |
| 203287_at | NM_005558.1 | Ladinin 1 | 4.74 | 2.06 × 10-05 |
| 204913_s_at | AI360875 | SRY (sex determining region Y)- | 4.77 | 2.44 × 10-05 |
| 217028_at | AJ224869 | | 4.82 | 2.56 × 10-05 |
| 204750_s_at | BF196457 | Desmocollin 2 | 4.84 | 1.78 × 10-05 |
| 216222_s_at | AI561354 | Myosin X | 4.84 | 1.93 × 10-05 |
| 1438_at | X75208 | EphB3 | 5.02 | 9.02 × 10-06 |
| 203693_s_at | NM_001949.2 | E2F transcription factor 3 | 5.17 | 4.83 × 10-06 |
| 205548_s_at | NM_006806.1 | BIG family, member 3 | 5.64 | 1.96 × 10-06 |
| 201976_s_at | NM_012334.1 | Myosin X | 5.68 | 8.74 × 10-07 |
| 213134_x_at | AI765445 | BlG family, member 3 | 5.76 | 1.31 × 10-06 |
| 40016g_at | AB002301 | KIAA0303 protein | 4.26 | 1.071 × 10-04 |
| 206352_s_at | AB013818 | peroxisome biogenesis factor 10 | 4.28 | 5.79 × 10-05 |
| 205074_at | AB015050 | solute carrier family 22 member 5 | 4.64 | 2.24 × 10-05 |
| 213527_s_at | AC002310 | similar to hypothetical protein | 4.62 | 3.16 × 10-05 |
| | | MGC13138 | | |
| 216835_s_at | AF035299 | docking protein 1,62 kDa | 4.44 | 3.32 × 10-05 |
| 209617_s_at | AF035302 | catenin (cadherin-associated protein), | 5.16 | 1.7 × 10-06 |
| | | delta 2 (neural plakophilin-related arm- | | |
| | | repeat protein) | | |
| 208945_s_at | AF139131 | beclin 1 (coiled-coil, myosin-like BCL2 | 5.61 | 5.0 × 10-07 |
| | | interacting protein) | | |
| 222275_at | AI039469 | mitochondrial ribosomal protein S30 | 4.51 | 2.16 × 10-05 |
| 203929_s_at | AI056359 | microtubule-associated protein tau | 6.60 | 0.0 × 10-04 |
| 215552_s_at | AI073549 | Estrogen receptor 1 | 4.51 | 2.51 × 10-05 |
| 212956_at | AI348094 | KIAA0882 protein | 4.40 | 7.0 × 10-05 |
| 204913_s_at | AI360875 | SRY (sex determining region Y)-box 11 | −4.45 | 9.92 × 10-05 |
| 213855_s_at | AI500366 | lipase, hormone-sensitive | 4.17 | 1.08 × 10-04 |
| 212239_at | AI680192 | phosphoinositide-3-kinase, regulatory | 4.36 | 4.71 × 10-05 |
| | | subunit, polypeptide 1 (p85 alpha) | | |
| 203928_x_at | AI870749 | microtubule-associated protein tau | 5.91 | 8 × 10-08 |
| 214124_x at | AL043487 | FGFR1 oncogene partner | 5.18 | 3.1 × 10-06 |
| 212195_at | AL049265 | MRNA; cDNA DKFZp564F053 | 4.25 | 1.11 × 10-04 |
| 210222_s_at | BC000314 | reticulon 1 | 4.08 | 1.07 × 10-04 |
| 210958_s_at | BC003646 | KIAA0303 protein | 4.43 | 4.26 × 10-05 |
| 204863_s_at | BE856546 | interleukin 6 signal transducer (gp130, | 4.28 | 8.20 × 10-05 |
| | | oncostatin M receptor) | | |
| 213911_s_at | BF718636 | H2A histone family, member Z | −4.16 | 1.10 × 10-04 |
| 212207_at | BG426689 | thyroid hormone receptor associated | 6.06 | 1.0 × 10-07 |
| | | protein 2 | | |
| 209696_at | D26054 | fructose-1,6-bisphosphatase 1 | 4.29 | 9.21 × 10-05 |
| 209443_at | J02639 | serine (or cysteine) proteinase inhibitor, | 4.21 | 6.95 × 10-05 |
| | | clade A (alpha-1 antiproteinase, | | |
| | | antitrypsin), member 5 | | |
| 202862_at | NM_000137 | fumarylacetoacetate hydrolase | 4.34 | 5.59 × 10-05 |
| | | (fumarylacetoacetase) | | |
| 214440_at | NM_000662 | N-acetyltransferase 1 (arylamine N- | 4.24 | 6.75 × 10-05 |
| | | acetyltransferase) | | |
| 208305_at | NM_000926 | progesterone receptor | 4.15 | 8.19 × 10-05 |
| 202204_s_at | NM_001144 | autocrine motility factor receptor | 5.28 | 1.29 × 10-06 |
| 204862_s_at | NM_002S13 | non-metastatic cells 3, protein expressed | 4.30 | 8.95 × 10-05 |
| | | in | | |
| 202641_at | NM_004311 | ADP-ribosylation factor-like 3 | 4.24 | 9.46 × 10-05 |
| 200896_x_at | NM_004494 | hepatoma-derived growth factor (high- | −4.87 | 1.38 × 10-05 |
| | | mobility group protein 1-like) | | |
| 203071_at | NM_004636 | sema domain, immunoglobulin domain | 4.65 | 1.63 × 10-05 |
| | | (Ig), short basic domain, secreted, | | |
| | | (semaphorin) 3B | | |
| 205012_s_at | NM_005326 | hydroxyacylglutathione hydrolase | 4.60 | 3.62 × 10-05 |
| 204916_at | NM_005855 | receptor (calcitonin) activity modifying | 5.47 | 5.10 × 10-07 |
| | | protein 1 | | |
| 204792_s_at | NM_014714 | KIAA0590 gene product | 4.14 | 1.12 × 10-04 |
| 208202_s_at | NM_015288 | PHD finger protein 15 | 4.18 | 1.08 × 10-04 |
| 217770_at | NM_015937 | phosphatidylinositol glycan, class T | 4.33 | 5.43 × 10-05 |
| 218671_s_at | NM_016311 | ATPase inhibitory factor 1 | 4.18 | 9.04 × 10-05 |
| 219872_at | NM_016613 | hypothetical protein DKFZp434L142 | 4.10 | 1.03 × 10-04 |
| 219197_s_at | NM_020974 | signal peptide, CUB domain, EGF-like 2 | 5.43 | 6.8 × 10-07 |
| 203485_at | NM_021136 | reticulon 1 | 4.18 | 7.56 × 10-05 |
| 206936_x_at | NM_022335 | NADH dehydrogenase (ubiquinone) 1, | 4.28 | 6.46 × 10-05 |
| | | subcomplex unknown, 2, 14.5kDa | | |
| 220540_at | NM_022358 | potassium channel, subfamily K, | 4.68 | 1.32 × 10-05 |
| | | member 15 | | |
| 219438_at | NM_024522 | hypothetical protein FLJ12650 | 4.82 | 6.68 × 10-06 |
| 205696_s_at | 2674 U97144 | GDNF family receptor alpha 1 | 4.89 | 7.15 × 10-06 |

In addition to other know methods of cancer therapy, hormone therapies may be employed in the treatment of patients identified as having hormone sensitive cancers. Hormones, or other compounds that stimulate or inhibit these pathways, can bind to hormone receptors, blocking a cancer's ability to get the hormones it needs for growth. By altering the hormone supply, hormone therapy can inhibit growth of a tumor or shrink the tumor. Typically, these cancer treatments only work for hormone-sensitive cancers. If a cancer is hormone sensitive, a patient might benefit from hormone therapy as part of cancer treatment. Sensitive to hormones is usually determined by taking a sample of a tumor (biopsy) and conducting analysis in a laboratory.

Cancers that are most likely to be hormone-receptive include: Breast cancer, Prostate cancer, Ovarian cancer, and Endometrial cancer. Not every cancer of these types is hormone-sensitive, however. That is why the cancer must be analyzed to determine if hormone therapy is appropriate.

Hormone therapy may be used in combination with other types of cancer treatments, including surgery, radiation and chemotherapy. A hormone therapy can be used before a primary cancer treatment, such as before surgery to remove a tumor. This is called neoadjuvant therapy. Hormone therapy can sometimes shrink a tumor to a more manageable size so that it's easier to remove during surgery.

Hormone therapy is sometimes given in addition to the primary treatment—usually after—in an effort to prevent the cancer from recurring (adjuvant therapy). In some cases of advanced (metastatic) cancers, such as in advanced prostate cancer and advanced breast cancer, hormone therapy is sometimes used as a primary treatment.

Hormone therapy can be given in several forms, including: (A) Surgery—Surgery can reduce the levels of hormones in your body by removing the parts of your body that produce the hormones, including: Testicles (orchiectomy or castration), Ovaries (oophorectomy) in premenopausal women, Adrenal gland (adrenalectomy) in postmenopausal women, Pituitary gland (hypophysectomy) in women. Because certain drugs can duplicate the hormone-suppressive effects of surgery in many situations, drugs are used more often than surgery for hormone therapy. And because removal of the testicles or ovaries will limit an individual's options when it comes to having children, younger people are more likely to choose drugs over surgery. (B) Radiation—Radiation is used to suppress the production of hormones. Just as is true of surgery, it's used most commonly to stop hormone production in the testicles, ovaries, and adrenal and pituitary glands. (C) Pharmaceuticals—Various drugs can alter the production of estrogen and testosterone. These can be taken in pill form or by means of injection. The most common types of drugs for hormone-receptive cancers include: (1) Anti-hormones that block the cancer cell's ability to interact with the hormones that stimulate or support cancer growth. Though these drugs do not reduce the production of hormones, anti-hormones block the ability to use these hormones. Anti-hormones include the anti-estrogens tamoxifen (Nolvadex) and toremifene (Fareston) for breast cancer, and the anti-androgens flutamide (Eulexin) and bicalutamide (Casodex) for prostate cancer. (2) Aromatase inhibitors—Aromatase inhibitors (AIs) target enzymes that produce estrogen in postmenopausal women, thus reducing the amount of estrogen available to fuel tumors. AIs are only used in postmenopausal women because the drugs can't prevent the production of estrogen in women who haven't yet been through menopause. Approved AIs include letrozole (Femara), anastrozole (Arimidex) and exemestane (Aromasin). It has yet to be determined if AIs are helpful for men with cancer. (3) Luteinizing hormone-releasing hormone (LH-RH) agonists and antagonists—LH-RH agonists—sometimes called analogs—and LH-RH antagonists reduce the level of hormones by altering the mechanisms in the brain that tell the body to produce hormones. LH-RH agonists are essentially a chemical alternative to surgery for removal of the ovaries for women, or of the testicles for men. Depending on the cancer type, one might choose this route if they hope to have children in the future and want to avoid surgical castration. In most cases the effects of these drugs are reversible. Examples of LH-RH agonists include: Leuprolide (Lupron, Viadur, Eligard) for prostate cancer, Goserelin (Zoladex) for breast and prostate cancers, Triptorelin (Trelstar) for ovarian and prostate cancers and abarelix (Plenaxis).

One class of pharmaceuticals are the Selective Estrogen Receptor Modulators or SERMs. SERMs block the action of estrogen in the breast and certain other tissues by occupying estrogen receptors inside cells. SERMs include, but are not limited to tamoxifen (the brand name is Nolvadex, generic tamoxifen citrate); Raloxifene (brand name: Evista), and toremifene (brand name: Fareston).

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Material and Methods

Patients and Samples. Studies were conducted using different cohorts of samples: 132 patients (82 were ER-positive) from UT M.D. Anderson Cancer Center (MDACC) prior to pre-operative adjuvant chemotherapy, 18 patients from MDACC with metastatic (AJCC Stage IV) ER-positive breast cancer, 277 patients from three different institutions (109 from Oxford, UK; 87 from Guy's Hospital, London UK; 81 from Uppsala, Sweden) who were uniformly treated with adjuvant tamoxifen, and 286 patients (209 were ER-positive) with node-negative disease from a single institution who did not receive any systemic chemotherapy treatment. At MDACC, pre-treatment fine needle aspiration (FNA) samples of primary breast cancer were obtained using a 23-gauge needle and the cells from 1-2 passes were collected into a vial containing 1 ml of RNAlater™ solution (Ambion, Austin Tex.) and stored at −80° C. until use, whereas archival frozen samples were evaluated from resected, metastatic, ER-positive breast cancer. All patients signed an informed consent for voluntary participation to collect samples for research. At other institutions, fresh tissue samples of surgically resected primary breast cancer were frozen in OCT compound and stored at −80° C.

Patients in this study had invasive breast carcinoma and were characterized for estrogen receptor (ER) expression using immunohistochemistry (IHC) and/or enzyme immunoassay (EIA). Immunohistochemical (IHC) assay for ER was performed on formalin-fixed paraffin-embedded (FFPE) tissue sections or Camoy's-fixed FNA smears using the following methods: FFPE slides were first deparaffinized, then slides (FFPE or FNA) were passed through decreasing alcohol concentrations, rehydrated, treated with hydrogen peroxide (5 minutes), exposed to antigen retrieval by steaming the slides in tris-EDTA buffer at 95° C. for 45 minutes, cooled to room temperature (RT) for 20 minutes, and incubated with primary mouse monoclonal antibody 6F11 (Novacastra/Vector Laboratories, Burlingame, Calif.) at a dilution of 1:50 for 30 minutes at RT (Gong et al., 2004). The Envision method was employed on a Dako Autostainer instrument for the rest of the procedure according to the manufacturer's instructions (Dako Corporation, Carpenteria, Calif.). The slides were then counterstained with hematoxylin, cleared, and mounted. Appropriate negative and positive controls were included. The 96 breast cancers from OXF were ER-positive by enzyme immunoassay as previously described, containing>10 femtomoles of ER/mg protein (Blankenstein et al., 1987).

Estrogen receptor (ER) expression was characterized using immunohistochemistry (IHC) and/or enzyme immunoassay (EIA). IHC staining of ER was interpreted at MDACC as positive (P) if ≧10% of the tumor cells demonstrated nuclear staining, low expression (L) if <10% of the tumor cell nuclei stained, and negative (N) if there was no nuclear staining. Low expression (<10%) is reported in routine patient care as negative, but some of those patients potentially benefit from hormonal therapy (Harvey et al., 1999).

RNA extraction and gene expression profiling. RNA was extracted from the MDACC FNA samples using the RNAeasy Kit™ (Qiagen, Valencia Calif.). The amount and quality of RNA was assessed with DU-640 U.V. Spectrophotometer (Beckman Coulter, Fullerton, Calif.) and it was considered adequate for further analysis if the OD260/280 ratio was ≧1.8 and the total RNA yield was ≧1.0 μg. RNA was extracted from the tissue samples using Trizol (InVitrogen, Carlsbad, Calif.) according to the manufacturer's instructions. The quality of the RNA was assessed based on the RNA profile generated by the Bioanalyzer (Agilent Technologies, Palo Alto, Calif.). Differences in the cellular composition of the FNA and tissue samples have been reported previously (Symmans et al., 2003). In brief, FNA samples on average contain 80% neoplastic cells, 15% leukocytes, and very few (<5%) non-lymphoid stromal cells (endothelial cells, fibroblasts, myofibroblasts, and adipocytes), whereas tissue samples on average contain 50% neoplastic cells, 30% non-lymphoid stromal cells, and 20% leukocytes (Symmans et al., 2003). A standard T7 amplification protocol was used to generate cRNA for hybridization to the microarray. No second round amplification was performed. Briefly, mRNA sequences in the total RNA from each sample were reverse-transcribed with SuperScript II in the presence of T7-(dT)24 primer to produce cDNA. Second-strand cDNA synthesis was performed in the presence of DNA Polymerase I, DNA ligase, and Rnase H. The double-stranded cDNA was blunt-ended using T4 DNA polymerase and purified by phenol/chloroform extraction. Transcription of double-stranded cDNA into cRNA was performed in the presence of biotin-ribonucleotides using the BioArray High Yield RNA transcript labeling kit (Enzo Laboratories). Biotin-labeled cRNA was purified using Qiagen RNAeasy columns (Qiagen Inc.), quantified and fragmented at 94° C. for 35 minutes in the presence of 1× fragmentation buffer. Fragmented cRNA from each sample was hybridized to each Affymetrix U133A gene chip, overnight at 42° C. The U133A chip contains 22,215 different probe sets that correspond to 13,739 human UniGene clusters (genes). Hybridization cocktail was prepared as described in the Affymetrix technical manual. dCHIP Vi.3 (available via the internet at dchip.org) software was used to generate probe level intensities and quality measures including median intensity, % of probe set outliers and % of single probe outliers for each chip.

Microarray Data Analysis. The raw intensity files (CEL) from each microarray were normalized using dChip V1.3 software (dchip.org). After normalization, the 75th percentile of pixel level was used as the intensity level for each feature on a microarray (see mdanderson.org/pdf/biostats_utmdabtr00503.pdf via the world wide web). Multiple features representing each probe set were aggregated using the perfect match model to form a single measure of intensity.

Definition of ER Reporter Genes. ER reporter genes were defined from an independent public dataset of Affymetrix U133A transcriptional profiles from 286 node-negative breast cancer samples (Wang et al., 2005). Expression data had been normalized to an average probe set intensity of 600 per array (Wang et al., 2005). The dataset was filtered to include 9789 probe sets with most variable expression, where P₀≧5, P₇₅−P₂₅≧100, and P₉₅/P₅≧3 (P_{q} is the q^{th} percentile of intensity for each probe set). Those were ranked by Spearman's rho (Kendall and Gibbons, 1990) with ER mRNA (ESR1 probe set 205225_at) expression, of which 2217 probe sets were significantly and positively associated with ESR1 (t-test of correlation coefficients with one-sided significance level of 99.9% and estimated false discovery rate (FDR) of 0.45%). The size of the reporter gene set was then determined by a bootstrap-based method that accounts for sampling variability in the correlation coefficient and in the resulting probe sets rankings (Pepe et al., 2003). The entire dataset was re-sampled 1000 times with replacement at the subject level (i.e., when one of the 286 subjects was selected in the bootstrap sample, the 2217 candidate probe sets from that subject were included in the dataset). Each probe set was ranked according to its correlation with ESR1 in each bootstrap dataset. The probability (P) of selection for each probe set (g) in a reporter gene set of defined length (k) was calculated as P[Rank(g)≦k]. A similar computation provided estimates of the power to detect the truly co-expressed genes from a study of a given size (Pepe et al., 2003).

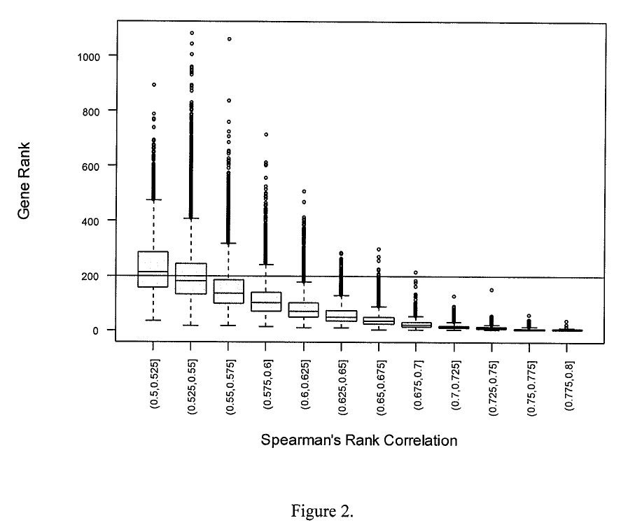

Genes that are truly co-expressed with ESR1 have selection probabilities close to 1, but the selection probability diminishes quickly for lower order probe sets (FIG. 1). The probability of selecting the top 50 ER-associated probes would be 98.5% if the ER reporter gene list included 200 probes, 87.0% if 100 probes, and 41.3% if 50 probes (FIG. 1). An ER reporter list with 200 top-ranking probes would include the top 50 probes with 98.5% probability and the top 100 probes with about 93% probability (FIG. 1). The distribution of ranks is very tight for genes that are strongly correlated with ESR1 having median ranks close to 1 (FIG. 2). However, both the median rank and the variance of the distribution of ranks increase for genes that are moderately correlated with ESR1. The gene ranks for genes with Spearman's rho>0.65 are less than 200 with the exception of a few outliers (FIG. 2). Therefore as opposed to selecting the reporter genes by choosing an arbitrary cutoff on the correlation coefficient, this approach identifies the 100 genes that are most-strongly correlated with ESR1 with high power (>93%). The size of the reporter gene set was selected to be 200 probe sets, based on the bootstrap-estimated selection probabilities (FIG. 1) and the requirement to detect the top 100 truly co-expressed genes with >90% power. The original dataset was re-sampled with replacement at the subject level (i.e., when one of the 286 subjects was selected in the bootstrap sample, the 2217 candidate probe sets from that subject were included in the dataset to generate 1000 different bootstrap datasets. Each candidate probe set was ranked according to its correlation with ESR1 within each bootstrap dataset and the degree of confidence in the ranking of each probe set was quantified in terms of the selection probability, Pg(k). The probability (P) of selection for each probe set (g) in a reporter gene set of defined length (k) was calculated as P[Rank(g)]≦k.

Calculation of Expression Index (Sensitivity to Endocrine Treatment Index). To quantify the expression of the 200 reporter genes in new samples, the inventors first developed a gene-expression-based ER associated index. ER-positive and ER-negative reference signatures, or centroids, were then described as the median log-transformed expression value of each of the 200 reporter genes in the 209 ER-positive and 77 ER-negative subjects, respectively. For new samples, the similarity between the log-transformed 200-gene ER associated gene expression signature with the reference centroids was determined based on Hoeffding's D statistic (Hollander and Wolfe, 1999). D takes into account the joint rankings of the two variables and thus provides a robust measure of association that, unlike correlation-based statistics, will detect nonmonotonic associations (in statistical terms, it detects a much broader class of alternatives to independence than correlation-based statistics). The ER reporter index (RI) was defined as the difference between the similarities with the ER+ and ER− reference centroids: RI=D⁺−D⁻.

The 200-gene signature of a tumor with high ER-dependent transcriptional activity resembles more closely the ER-positive centroid and therefore D⁺ will be greater than D⁻ and RI will be positive. The opposite will be the case for tumors with low ER-related activity and thus RI will be small or negative. Subtraction of D normalizes the reporter index relative to the basal levels of expression of the ER-related genes in ER negative tumors. Because of this and since D is a distribution-free statistic, RI is relatively insensitive to the method used to normalize the microarray data and therefore can be computed across datasets. From the RI, a genomic index of sensitivity to endocrine therapy (SET) was calculated as follows: SET=100(RI+0.2)³. The offset translated RI to mostly positive values and was then transformed to normality using an unconditional Box-Cox power transformation. Finally, the maximum likelihood estimate of the exponent was rounded to the closest integer and the index was scaled to a maximum value of 10.

Statistical Analysis of Distant relapse-free survival (DRFS). Distant relapse-free survival (DRFS) was defined as the interval from breast surgery until diagnosis of distant metastasis. Covariate effects on distant relapse risk after tamoxifen treatment were evaluated using log-rank test in multivariate Cox proportional hazards models stratified by institution. The covariates we included were genomic measurement of likely sensitivity to endocrine therapy (SET index), gene expression levels of estrogen receptor (ESR1, probe set 205225) and progesterone receptor (PGR, probe set 208305), age at diagnosis, tumor histologic grade and tumor stage (revised American Joint Committee on Cancer (AJCC) staging system). ESR1 was normally distributed, but PGR levels were log-transformed to normality. To determine the continuous relation between the SET index and 10-year DRFS, the data were fitted by Cox proportional hazards models having a smoothing spline approximation with 2 degrees of freedom of the SET index as the only covariate (Therneau and Grambsch, 2000). The baseline cumulative hazard rate was estimated from the Cox model based on the Nelson-Aalen estimator and the predicted rate of distant relapse was then obtained from the Breslow-type estimator of the survival function. Confidence intervals of the survival estimate were calculated based on the Tsiatis variance estimates of the cumulative log-hazards (Therneau and Grambsch, 2000). A similar approach was used to determine the continuous relation between ESR1 and PGR expression and DRFS.

Likely sensitivity to endocrine therapy was classified as low, intermediate, or high using cutoff points of the SET index values determined by fitting on the entire dataset (n=277) a stratified multivariate Cox model to predict DRFS in relation to age, histologic grade, stage, median-dichotomized ESR1, median-dichotomized PGR, and the trichotomous SET indicator variable using different thresholds. Thresholds that resulted in maximum or near maximum log-profile likelihood for this model were selected as most informative cut points for predicting DRFS (Tableman and Kim, 2004). The same thresholds were maintained for subsequent analyses of the untreated patients. All statistical computations were performed in R(R Development Core Team, 2005).

### Example 2

### Correlation Between ER mRNA Expression Levels and ER Status

Intensity values of ESR1 (ER) gene expression from microarray experiments were compared to the results from standard IHC and enzyme immunoassays in 82 FNA samples (MDACC). The Affymetrix U133A GeneChip™ has six probe sets that recognize ESR1 mRNA at different sequence locations. A comparison of the different probe sets using the 82 FNA dataset is presented in Table 3. All the ESR1 probe sets showed high correlation with ER status determined by immunohistochemistry (Kruskal-Wallis test, p<0.0001). The probe set 205225_had the highest mean, median, and range of expression and was most correlated with ER status (Spearman's correlation, R=0.85, Table 3).

TABLE 3
The mean, median, and range of expression of the six probe sets that
identify ERα gene (ESR1) are compared using the results from 82 FNA
samples. Expression of each ESR1 probe set is correlated to ER status
(positive, low, or negative) and to the expression of the ESR1 205225_{—}
probe set (R values, Spearman‘s rank correlation test).

| Probe Set | | | | Spearman | |
|---|---|---|---|---|---|
| ER | Signal Intensity | | | Correlation With | |
| ESR1 | Mean | Median | Range | ER Status | 205225_{—} |
| 205225_{—} | 1633 | 912 | 6802 | 0.85 | 1.00 |
| 215552_{—} | 192 | 136 | 671 | 0.81 | 0.86 |
| 217190_{—} | 152 | 122 | 429 | 0.72 | 0.84 |
| 211233_{—} | 234 | 178 | 663 | 0.71 | 0.88 |
| 211235_{—} | 189 | 139 | 674 | 0.69 | 0.88 |
| 211234_{—} | 236 | 209 | 462 | 0.64 | 0.83 |

### Example 3

### ER Reporter Genes

The consistency of identifying top-ranking genes depends on factors that affect the sampling variability in the correlation coefficient, such as the size of the dataset and the strength of the underlying true association between the candidate genes and ESR1. The inventors evaluated the consistency in the ranking of the candidate ER reporter genes in terms of the selection probability estimated from 1000 bootstrapped datasets. FIG. 1 shows that the selection probability was high for the top-ranking probes, i.e., the top-ranking probes rank consistently at the top of the list, but it diminished quickly with increasing rank. Furthermore, the selection probability of a candidate gene of a given rank showed a strong dependence on the number of candidate probes selected. For example, the probability of consistently selecting the truly top 50 ER-associated probes was 98.5% if the top 200 candidate probes are selected, 87.0% if the top 100 probes are selected, and only 41.3% if the top 50 probes are selected (FIG. 1). Based on these considerations, the inventors defined the ER reporter list to include the 200 top-ranking probes to ensure that the 100 most-strongly associated probes with ESR1, which are expected to be biologically relevant, would be among the reporter genes with about 90% probability. The entire list included 200 probe sets (excluding those that detect ESR1) representing 163 different genes and 7 uncharacterized transcripts (Table 1).

### Example 4

### ER Reporter Index is Independent of ESR1 Expression

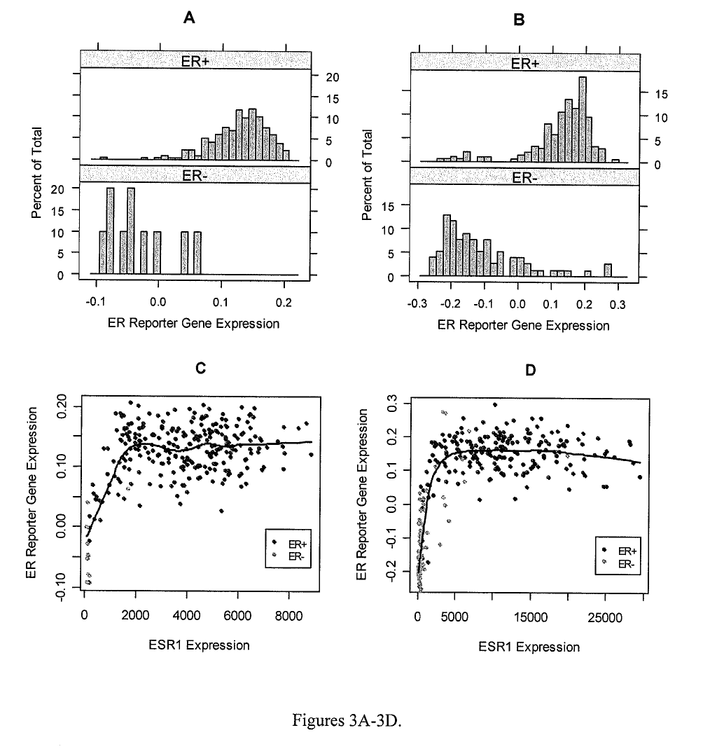

The ER reporter index (RI) was calculated for the tamoxifen-treated group and the node-negative untreated group. The RI was predominantly positive in ER-positive subjects and predominately negative in ER-negative subjects with the two ER-conditional distributions being distinct and well separated (FIGS. 3A and 3B), which supports ER RI as an indicator of ER-associated activity. To evaluate whether the levels of ER RI are correlated with ESR1 mRNA expression levels, the RI was plotted vs. ESR1 expression for both groups (FIGS. 3C and 3D). Although both ESR1 mRNA and RI were lower in ER-negative subjects, there was no apparent trend in ER-positive subjects. This suggests that, even though the estrogen reporter genes were identified as being co-expressed with ESR1, the overall expression pattern of this group of genes as captured by the ER reporter index conveys information on ER-signaling that is not captured by ESR1.

### Example 5

### Reproducibility of Reporter Genes and SET Index

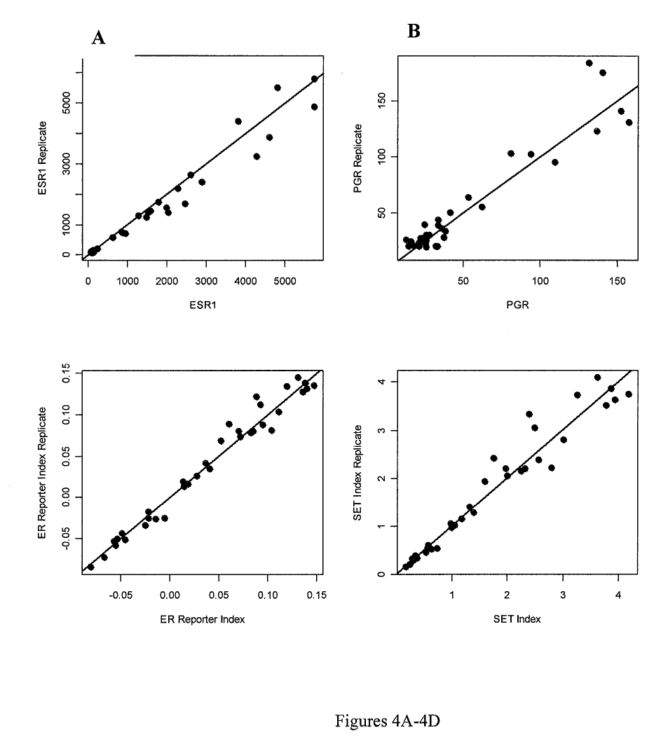

The in vivo transcription and microarray hybridization steps were repeated using residual sample RNA from 35 FNA samples. The 35 original and replicate sample pairs demonstrated excellent reproducibility of the gene expression measurements and calculated indices (FIG. 4). The concordance correlation coefficients were (Lin, 1989; 2000): 0.979 (95% CI 0.958-0.989) for the pairs of ESR1 expression measurements, 0.953 (95% CI 0.909-0.976) for PGR expression, 0.985 (95% CI 0.972-0.992) for ER reporter index values, and 0.972 (95% CI 0.945-0.986) for the pairs of SET index measurements exhibiting excellent accuracy (minimal deviation of the best fit line from the 45° line) and good precision in all cases.

### Example 6

### Characterization of ER Reporter Genes

The 200 ER reporter probe sets represent 163 unique genes and 7 uncharacterized transcripts (Table 1). These contain twenty-seven probe sets that represent 23 genes on chromosome 5, and 20 probe sets that represent 18 genes on chromosome 1. Mapping the 163 genes to the KEGG pathway database indicated representation of several signaling pathways including focal adhesion, Wnt, Jak-STAT, and MAPK signaling pathways. Furthermore, mapping to gene ontology (GO) categories indicated that the biological processes “fatty acid metabolism,” “pyrimidine ribonucleotide biosynthesis,” and “apoptosis” are over-represented in this set relative to chance based on the hypergeometric test (p-values<0.03). The distributions of reporter genes for ER-positive and ER-negative breast cancers were distinct and well separated, consistent with an indicator of ER-associated activity (FIGS. 3A and 3B). Both ESR1 and reporter genes were lower in ER-negative subjects, but there was no apparent correlation in ER-positive subjects (FIGS. 3C and 3D). Therefore, although the ER reporter genes were identified by their co-expression with ESR1, the overall expression pattern of this group of genes (as captured by the index) conveys information on ER-signaling that is independent of ER gene expression level alone.

### Example 7

### Distant Relapse after Adjuvant Tamoxifen Therapy

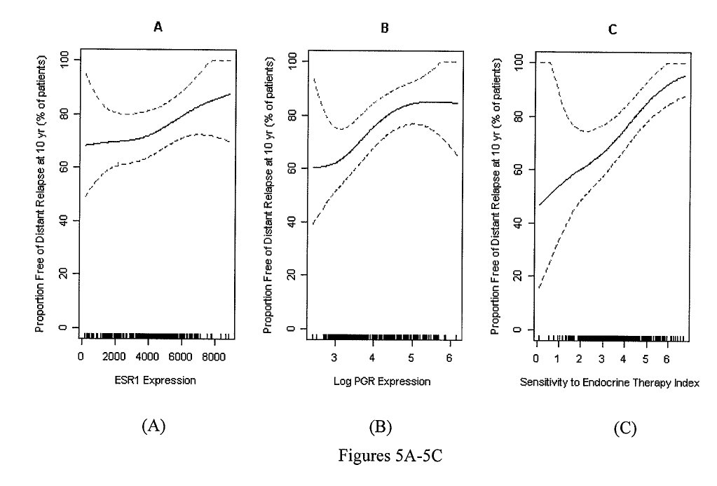

Univariate Cox proportional hazards models were employed to evaluate the risk of distant relapse at 10 years after adjuvant tamoxifen treatment as continuous functions of expression levels of the estrogen receptor gene (ESR1), progesterone receptor gene (PGR), and the 200-gene index of reporter genes for sensitivity to endocrine therapy (SET index) (FIG. 5). ER gene expression (ESR1, FIG. 5A) was not a significant predictor of 10-year relapse rate (LRT p=0.16), but higher progesterone receptor gene expression (PGR, FIG. 5B) was significantly associated with lower relapse rates at 10 years (HR 0.62; 95% CI 0.44-0.88; LRT p=0.005). Higher SET index levels (FIG. 5C) were also significantly associated with lower 10-year relapse rates (HR 0.70; 95% CI 0.56-0.86; LRT p<0.001). The mean relapse-free survival at 10 years for subjects with SET index<2 was 57.1% (95% CI 41.1-80.3) whereas for those with SET index>5 was 90.0% (95% CI 82.5-97.7) (FIG. 5C).

### Example 8

### Distant Relapse in Untreated Patients—SET Index is Independent of Prognosis

To address the possibility that observed differences in DRFS could be due to indolent prognosis, rather than benefit from adjuvant tamoxifen, the same covariates were evaluated as potential prognostic factors of DRFS in 209 ER-positive patients who did not receive adjuvant systemic therapy. Consistent with the effects in the tamoxifen treated group, ER expression level (ESR1, FIG. 6A) was not significantly associated with the 5-year relapse rate in untreated patients (LRT p=0.75), and higher progesterone receptor (PGR, FIG. 6B) was significantly associated with lower relapse rates at 5 years (HR 0.78, 95% CI 0.67-0.90; LRT p<0.001). However, the effect of the SET index (FIG. 6C) on the 5-year relapse rate in untreated patients was small and marginally significant (HR 0.90, 95% CI 0.82-1.00; LRT p=0.043).

### Example 9

### Independence of Genomic Predictors in Multivariate Survival Analyses

The continuous gene-expression-based predictors (ESR1, PGR, and SET index) were evaluated in a multivariate Cox model in relation to patient's age, tumor histologic grade and tumor AJCC stage for ER-positive patients treated with adjuvant tamoxifen. SET index was a significant predictor of relapse after adjuvant tamoxifen treatment (HR 0.72; 95% CI 0.54-0.95), whereas the effect of PGR expression was not statistically significant (Table 4, Treated Patients). Conversely, when patients with ER-positive breast cancer who did not receive adjuvant treatment were evaluated with the same multivariate model, it was found that PGR expression was independently prognostic (HR 0.72; 95% CI 0.58-0.89), whereas the effect of SET index was not statistically significant (Table 4, Untreated Patients). Therefore the SET index was independently predictive of benefit from adjuvant tamoxifen therapy, but not prognostic in patients with ER-positive breast cancer who did not receive adjuvant treatment.

TABLE 4
Multivariate Cox analysis of continuous gene-expression-based covariates of
DRFS in patients with ER-positive breast cancer. Treated patients (left column) received
adjuvant tamoxifen, whereas untreated patients (right column) had node-negative disease
and did not receive adjuvant treatment. ‡PGR expression values were log-transformed.

| | Treated Patients (n=211) | | Untreated Patients (n=142) | |
|---|---|---|---|---|
| Effect | HR (95% CI) | P-value | HR (95% CI) | P-value |
| Age | 1.09 (0.30-3.90) | 0.89 | 0.59 (0.31-1.11) | 0.10 |
| >50 vs. ≦ 50 | | | | |
| Histologic Grade | 1.09 (0.54-2.22) | 0.81 | 1.93 (0.92-4.04) | 0.08 |
| 3 vs. 1 or 2 | | | | |
| AJCC Stage | 1.96 (0.80-4.78) | 0.14 | 1.13 (0.64-1.97) | 0.68 |
| II or III vs. I | | | | |
| ER Expression | 1.00 (1.00-1.00) | 0.72 | 1.00 (1.00-1.00) | 0.13 |
| PGR Expression | 0.93 (0.61-1.40) | 0.72 | 0.72 (0.58-0.89) | 0.002 |
| Sensitivity to Endocrine | 0.72 (0.54-0.95) | 0.022 | 0.99 (0.86-1.14) | 0.86 |
| Therapy Index | | | | |

The SET index was developed to measure ER-related gene expression in breast cancer samples with a hypothesis that this would represent intrinsic endocrine sensitivity. The inventors found that SET index had a steep and linear association with improved 10-year relapse-free survival in women who received tamoxifen as their only adjuvant therapy (FIG. 2), and was the only significant factor in multivariate analysis of DRFS that included grade, stage, age, and expression levels of ESR1 and PGR (Table 4). The information from SET index is mostly predictive of benefit from endocrine treatment, rather than prognosis (FIG. 6, Table 4).

### Example 10

### Classes of Endocrine Sensitivity Defined By Set Index

The almost linear functional dependence of the likelihood of distant relapse on the genomic endocrine sensitivity (SET) index (FIG. 5C) makes it possible to define three classes by specifying two cut points. Optimal thresholds were chosen to maximize the predictability of the trichotomous SET index in a multivariate Cox model, and occurred at the 50^{th} and 65^{th} percentiles of SET distribution corresponding to index values 3.71 and 4.23, respectively. The three classes of predicted sensitivity to endocrine therapy (low, intermediate, and high sensitivity) were evaluated in a multivariate Cox model stratified by institution that included dichotomized age, histologic grade, AJCC stage, and the median-dichotomized gene expression of ESR1 and PGR. The likelihood of distant relapse after tamoxifen therapy was significantly lower in those in the high SET group, compared with the low SET group (HR=0.24, 95% CI 0.09-0.59, p=0.002). There was no significant difference between intermediate and low SET groups (HR=0.67; 95% CI 0.30-1.49; p=0.33).

### Example 11

### SET Index and Classes Correlate with Distant Relapse-Free Survival

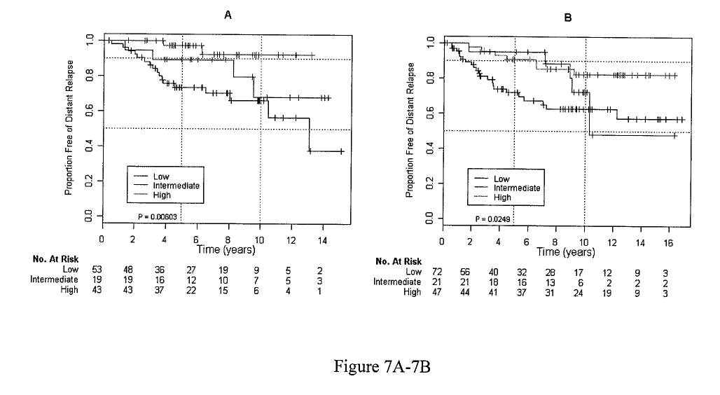

Kaplan-Meier estimators of relapse-free survival were compared for the three classes of SET index in the patients with ER-positive breast cancer who received adjuvant tamoxifen (FIG. 7A) with those who did not receive adjuvant therapy (FIG. 7B). The 35% of subjects with high SET had improved and sustained survival benefit from adjuvant tamoxifen, whereas the 50% of subjects with low SET did not obtain as much benefit from adjuvant tamoxifen (FIG. 7A). Most interesting were the 15% of subjects with intermediate SET. In the untreated cohort (FIG. 7B), subjects with intermediate SET had similar prognosis to those with low SET. However, in the tamoxifen treated cohort (FIG. 7A), subjects with intermediate SET had similar prognosis to those with high SET for the first 6 to 7 years of follow up. Furthermore, within 2 years after the completion of endocrine therapy these patients with intermediate SET began to experience distant relapse at a rate that was similar to the low SET group during the first 3 to 4 years of follow up (FIGS. 7A and 7B). Finally, the Kaplan-Meier estimators of relapse-free survival based on PGR expression (FIGS. 3C and 3D) confirm the combined prognostic and predictive effects of PGR (also shown in FIGS. 5B and 6B) and demonstrate less pronounced separation of the survival curves than SET in tamoxifen treated subjects (FIGS. 7A and 7C).

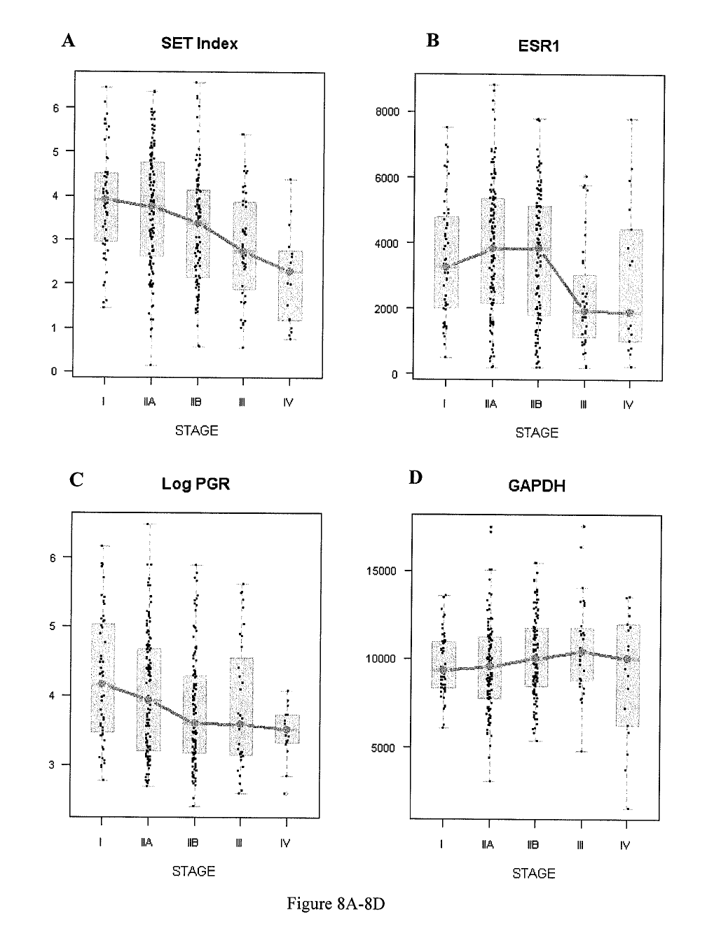

The inventors observed the same effects of SET class on DRFS of patients treated with adjuvant tamoxifen when the inventors stratified this cohort by known nodal status and separately evaluated the three classes of SET index in 115 node-negative patients (FIG. 8A) and 140 node-positive patients (FIG. 8B). These three classes of SET appear to identify approximately 35% of patients who have sustained benefit from adjuvant tamoxifen alone, approximately 50% who have minimal benefit from tamoxifen, and approximately 15% of patients whose benefit from tamoxifen continues during their adjuvant treatment, but is not sustained after endocrine therapy is completed.

Patients with high endocrine sensitivity (SET index values in upper 35%) had sustained benefit from adjuvant tamoxifen, compared to untreated patients (FIG. 7). This effect was evident when comparing untreated prognosis with tamoxifen treatment in node-negative patients (FIGS. 7B and 8A). Rare relapse events during tamoxifen treatment might still occur because of individual differences in compliance, metabolism due to variant genotype of cytochrome p450 2D6, or interaction from selective serotonin reuptake inhibitors used as antidepressants or to treat hot flashes. These can limit metabolism of tamoxifen to more active metabolites, thereby decreasing treatment efficacy, and are obviously unrelated to the activity of ER in the breast cancer cells (Stearns et al., 2003; Jin et al., 2005). Patients with low SET index values (lower 50%) derived minimal benefit from adjuvant tamoxifen, irrespective of nodal status (FIGS. 11 and 12). The effect of adjuvant tamoxifen (compared to untreated prognosis) is particularly revealing for patients with intermediate SET index (FIG. 7). These patients derived benefit from tamoxifen during their adjuvant treatment, but relinquished this survival benefit after cessation of treatment. Subjects with intermediate SET index started to accrue distant relapse events within 2 years of discontinuing adjuvant tamoxifen, and at a rate that was similar to the subjects with low SET index (treatment or prognosis) in the early period of follow up. This suggests that intermediate SET index values identified patients who might benefit from prolonged and/or more effective endocrine therapy used in current crossover treatment strategies (Goss et al., 2003).

### Example 12

### SET Index and Chemotherapy Response in ER-Positive Breast Cancer

Groups with low, intermediate, and high SET index were compared with pathologic response outcome in the 82 patients with ER-positive breast cancer who received neoadjuvant chemotherapy with paclitaxel (12 weekly cycles) followed by fluorouracil, doxorubicin, and cyclophosphamide (4 cycles q3 weeks) (Ayers et al., 2004). The same SET classes were as for the survival analyses after adjuvant tamoxifen. There were 8 patients with ER-positive cancer who achieved pathologic complete response (pCR) in the breast and axilla, of which 7 had low SET and one had intermediate SET (Table 5). Conversely, none of the 11 patients with ER-positive breast cancer and high SET, and only one of 11 patients with intermediate SET, achieved pCR from neoadjuvant T/FAC chemotherapy (Table 5).

TABLE 5
Pathologic response to neoadjuvant T/FAC chemotherapy in ER-positive
patients compared with predicted sensitivity to endocrine therapy
(SET risk groups).

| | Chemotherapy Response (ER + patients) | |
|---|---|---|
| Sensitivity to | | |
| Endocrine Therapy | Compete Pathologic | |
| (SET) Group | Response | Residual Disease |
| Low | 7 | 53 |
| Intermediate | 1 | 10 |
| High | 0 | 11 |

### Example 13

### SET Index and Stage of ER-Positive Cancer

There was a progressive decline in the values for the sensitivity to endocrine therapy (SET) index with increasing AJCC stage of ER-positive breast cancers (FIG. 8A, p<0.001). The decrease is only marginally significant for the transcriptional levels of ESR1 (FIG. 8B, p=0.04) and PGR (FIG. 8C, p=0.05), whereas the transcriptional level of a housekeeper gene (GAPDH) does not vary with stage (FIG. 8D, p=0.77). This analysis was done for 351 breast cancers that were ER-positive by IHC and had known stage of disease at the time of sample (58 stage I, 123 stage IIA, 107 stage IIB, 44 stage III, and 18 stage IV). The significance of stage-related trends was evaluated by treating tumor stage as an ordinal covariate in ordinary least squares regression with orthogonal polynomial contrasts. The p-values correspond to the significance of the linear term (based on the t-test). All samples from Stage I to III breast cancer were collected prior to any treatment. The 18 samples of Stage IV ER-positive breast cancer were from relapsed disease in 17 patients and at the time of initial presentation in one, and these included 14 patients who had received previous hormonal treatment with tamoxifen and/or aromatase inhibition. There was no obvious difference in the genomic expression levels of ESR1 or SET index in the 14 patients with Stage IV breast cancer who had received prior hormonal therapy, compared to the 4 who had not (ANOVA p=0.9).

Stage-dependent differences in biomarker measurements have obvious clinical importance, particularly for biomarkers of critical targeted cellular pathways. SET index values successively declined with advancing stage, whereas changes in ESR1 and PGR were less distinct (FIG. 8). One explanation is that tumors with less intrinsic dependence on estrogen are more biologically aggressive, and hence more likely to present with larger size and nodal metastasis. Additionally, biological progression of ER-positive breast cancer probably includes progressive dissociation from estrogen dependence through recruitment of other growth and survival pathways. The SET index captures these important differences in tumor biology with greater acuity than measurements of ER and PR. If significant decrease in genomic SET index values between matched primary tumors and subsequent distant metastases were demonstrated, then SET index could be used to monitor changes in the ER genomic pathway (and endocrine sensitivity) during the course of disease.

### REFERENCES

The following references, to the extent that they provide exemplary procedural or other details supplementary to those set forth herein, are specifically incorporated herein by reference.
U.S. Pat. No. 6,673,914
U.S. Pat. No. 6,521,415
U.S. Pat. No. 6,162,606
U.S. Pat. No. 6,107,034
U.S. Pat. No. 5,693,465
U.S. Pat. No. 5,384,260
U.S. Pat. No. 5,292,638
U.S. Pat. No. 5,030,417
U.S. Pat. No. 4,968,603
U.S. Pat. No. 4,806,464

### OTHER REFERENCES

Ayers et al., J. Clin. Oncol., 22:2284-2293, 2004.
Blankenstein et al., Clin. Chim. Acta, 165L189-195, 1987.
Bonneterre et al., J. Clin. Oncol., 18:3748-57, 2000.
Bryant and Wolmark, N. Engl. J. Med., 349(19):1855-1857, 2003.
Burstein, N. Engl. J. Med., 349(19):1857-1859, 2003.
Buzdar, Semin. Oncol., 28:291-304, 2001.
Esteva et al., Clin. Cancer Res., 11:3315-9, 2005.
Gong et al., Cancer, 102:34-40, 2004.
Goss et al., N. Engl. J. Med., 349(19):1793-1802, 2003.
Gruvberger-Saal et al., Mol. Cancer. Ther., 3:161-168, 2004.
Gruvberger et al., Cancer Res., 61:5979-5984, 2001.
Harvey et al., J. Clin. Oncol., 17:1474-1481, 1999.
Hess et al., Breast Cancer Res. Treat., 78:105-118, 2003.
Hollander and Wolfe, In: Probability and Statistics, Wiley Series, NY: John Wiley & Sons, Inc., 1999.
Howell and Dowsett, Breast Cancer Res., 6:269-274, 2004.
Howell et al., Lancet., 365(9453):60-62, 2005.
Jansen et al., J. Clin. Oncol., 23:732-740, 2005.
Jin et al., J. Natl. Cancer Inst., 97(1):30-39, 2005.
Kendall and Gibbons, In: Rank Correlation Methods, NY, Oxford University Press, 1990.
Konecny et al., J. Natl. Cancer Inst., 95:142-153, 2003.
Kun et al., Hum. Mol. Genet., 12:3245-3258, 2003.
Lacroix et al., Breast Cancer Res. Treat., 67:263-271, 2001.
Loi et al., Proc. Am. Soc. Clin. Oncol., Abstract #509, 2005
Ma et al., Cancer Cell, 5:607-616, 2004.
Mouridsen et al., J. Clin. Oncol., 19:2596-2606, 2001.
Paik et al., N. Engl. J. Med., 351:2817-2826, 2004.
Paik et al., Proc. Am. Soc. Clin. Oncol., Abstract #510, 2005.
Pepe et al., Biometrics, 59:133-142, 2003.
Perou et al., Nature, 406:747-752, 2000.
Pusztai et al., Clinical Cancer Res., 9:2406-2415, 2003.
Ransohoff, Nat. Rev. Cancer, 4:309-314, 2004.
Ransohoff, Nat. Rev. Cancer, 5:142-149, 2005.
Regitnig et al., Virchows Arch., 441:328-34, 2002.
Rhodes et al., J. Clin. Pathol., 53:125-130, 2000.
Rhodes, Am. J. Surg. Pathol., 27(9):1284-1285, 2003.
Rudiger et al., Am. J. Surg. Pathol., 26:873-882, 2002.
Sorlie et al, Proc. Natl. Acad. Sci. USA, 98:10869-10874, 2001.
Stearns et al., J. Natl. Cancer Inst., 95(23):1758-1764, 2003.
Symmans et al., Cancer, 97:2960-2971, 2003.
Tableman and Kim, In: Survival Analysis Using S: Analysis of Time-to-Event Data, FL,: Chapman & Hall/CRC; 2004.
Taylor et al., Hum. Pathol., 25:263-270, 1994.
Therneau and Grambsch, In: Modeling Survival Data: Extending the Cox Model, NY, Springer-Verlag; 2000.
Thurlimann et al., N. Engl. J. Med., 353(26):2747-2757, 2005.
van't Veer et al., Nature, 415:530-536, 2002.
Wang et al., Lancet., 365:671-679, 2005.

1. A method of assessing cancer patient sensitivity to treatment comprising the step of preparing a sensitivity to endocrine therapy (SET) index based on expression in a patient sample of one or more ER-related genes selected from Table 1.

2. The method of claim 1, further comprising selecting a treatment based on the SET index.

3. The method of claim 1, wherein the ER-related genes comprise 25 or more ER related genes of Table 1.

4. The method of claim 3, wherein the ER-related genes comprise 50 or more ER related genes of Table 1.

5. The method of claim 4, wherein the ER-related genes comprise 100 or more ER related genes of Table 1.

6. The method of claim 4, wherein the ER-related genes comprise 200 ER related genes of Table 1.

7. The method of claim 1, wherein the SET index includes covariates of tumor size, nodal status, grade, and age.

8. The method of claim 1, wherein the SET index includes evaluation of overall survival (OS).

9. The method of claim 8, wherein the SET index includes evaluation of distant relapse-free survival (DRFS).

10. The method of claim 1, wherein the treatment is a combination of one or more cancer therapy.

11. The method of claim 1, wherein the treatment is hormonal therapy.

12. The method of claim 11, wherein the hormonal therapy is tamoxifen therapy, aromatase inhibitor therapy, or SERM therapy.

13. The method of claim 11, wherein the treatment is chemotherapy.

14. The method of claim 11, wherein the treatment is a combination of hormonal therapy and chemotherapy.

15. The method of claim 1, wherein the patients are diagnosed with early or late-stage cancer.

16. A method of calculating a sensitivity to endocrine treatment (SET) index comprising the steps of:
(a) identifying a gene set of one or more estrogen receptor (ER)-related genes indicative of ER transcriptional activity by assessing gene expression in a reference population of tumor samples from cancer patients, defining a reference ER-related gene set; and
(b) preparing a calculated index using an assessment of ER-related gene expression in one or more samples relative to the reference ER-relate gene expression.

17. The method of claim 16, further comprising assessing sensitivity of a cancer to therapy using the calculated index.

18. The method of claim 17, wherein the therapy is hormonal therapy or chemotherapy.

19. The method of claim 18, wherein the therapy comprises both hormonal therapy and chemotherapy.

20. The method of claim 19, further comprising selecting a class or individual hormonal therapy.

21. The method of claim 20, wherein the hormonal therapy is tamoxifen therapy, aromatase inhibitor therapy, or SERM therapy.

22. The method of claim 17, further comprising identifying a patient that will benefit from an extended duration of therapy.

23. The method of claim 16, wherein all or part of the reference tumor samples are from patients diagnosed with a hormone sensitive cancer.

24. The method of claim 23, wherein the hormone sensitive cancer is an estrogen sensitive cancer.

25. The method of claim 24, wherein the estrogen-sensitive cancer is breast cancer.

26. The method of claim 16, wherein the gene set comprises 25 to 200 ER related genes.

27. The method of claim 26, wherein the gene set comprises 50 to 200 ER related genes.

28. The method of claim 27, wherein the gene set comprises 200 ER related genes.

29. The method of claim 16, wherein the calculated index includes a metric indicative of ER status of all or part of the reference tumor samples.

30. The method of claim 16, wherein the calculated index includes covariates of tumor size, nodal status, grade, and age.

31. The method of claim 16, wherein the calculated index includes evaluation of survival of the patient population sampled for all or part of the reference population of tumor samples.

32. The method of claim 31, wherein calculation of the index includes evaluation of distant relapse-free survival (DRFS) of the patient population.

33. The method of claim 16, wherein the patient population include ER-positive or both ER positive and ER negative samples.

34. The method of claim 16, further comprising normalizing expression data of the one or more samples to the ER-related gene expression profile.

35. The method of claim 34, wherein the expression data is normalized to a digital standard.

36. The method of claim 35, wherein the digital standard is a gene expression profile from a reference sample.

37.-42. (canceled)
